(19) United States
(12) Patent Application Publication
(10) Pub. No.: US 2006/0280799 A1
(43) Pub. Date: Dec. 14, 2006

(54) Carrier particles

(75) Inventors: Nicola Tirelli, Manchester (GB); Francesco Cellesi, Manchester (GB)

Correspondence Address: DAVIS WRIGHT TREMAINE LLP, 865 FIGUEROA STREET, SUITE 2400, LOS ANGELES, CA, 90017-2566, US

(73) Assignee: The University of Manchester, Manchester (GB)

(21) Appl. No.: 10/555,991

(22) PCT Filed: May 21, 2004
(86) PCT No.: PCT/GB04/02196

§ 371 Date: Nov. 8, 2005

(30) Foreign Application Priority Data
May 21, 2003 (GB) 03116647

(51) Int. Cl. A61K 9/14

(52) U.S. Cl. 424/489; 977/906

(57) ABSTRACT

A carrier particle (10) is arranged in use to encapsulate and carry a payload molecule (4) to a target biological environment, and comprises an internal cavity (8) in which a payload molecule (4) is contained. The cavity (8) is surrounded by a perm-selective hydrogel layer (6), and the payload molecule (4) is capable of being active when the particle (10) is at least adjacent the target biological environment.

The present invention relates to carrier particles and particularly, although not exclusively, to carrier particles, which may be used for the encapsulation and delivery of sensitive biomolecules.

A number of vesicular carriers used to encapsulate, carry and deliver sensitive biomolecules or ‘payload’ molecules to target sites, exist. Examples include liposomes, niosomes, and polymeric vesicles etc. Such particles provide a water cavity-containing structure in which the payload biomolecule is contained. The payload molecule is surrounded by a liquid-like membrane, that is generally composed of amphiphilic compounds, for example, phospholipids. The carriers are capable of freely circulating in body fluids and may be used to deliver the payload biomolecule to a specific target cell or tissue.

A variety of compositions exist for vesicular carriers, which differ in the nature of the amphiphilic compound, i.e. the phospholipids, non-ionic low molecular weight amphiphilies, and block copolymers. In addition, the composition of their hydrophobic domain also differs, i.e. by the presence of stabilizing agents, such as cholesterol. In addition, the surface chemistry of the carrier can differ, for example, the surface of the vesicular carrier can display specific polymers for a minimisation of protein adsorption and a prolongation of the carrier life-time in vivo.

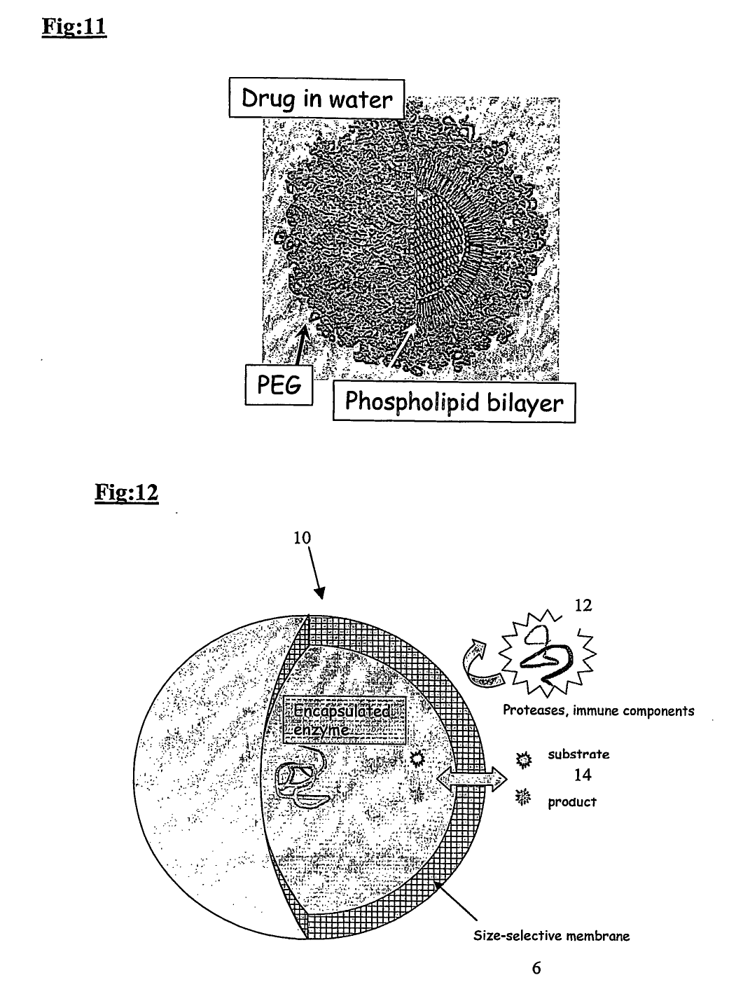

An example of such a carrier is a liposome displaying polyethylene glycol (PEG) on its surface, i.e. a PEGylated or a STEALTH® liposome, which is shown in FIG. 11. The liposome encapsulates a drug in the internal water cavity, and the membrane is composed of a bilayer of phospholipids, which are in part functionalised with PEG chains. These PEG polymer chains are essential in providing resistance to protein adsorption and thus reduce recognition of the carrier as a foreign body, which ultimately results in prolonged circulation times.

However, in such vesicular carriers, any encapsulated material is not in contact with the external environment, as long as the integrity of the carrier is preserved. Hence, the payload molecule is maintained in an inactive state until it is released only when the carrier has reached its final target, or it is disrupted by physical or mechanical action. At this target site, the released payload compound becomes active. However, after release of the encapsulated payload, the carrier membrane is no longer able to perform any protective action towards the active compound or payload. This can be problematic, as in many applications, it is often desirable that the active components perform their function when still protected by the carrier.

It is an aim of embodiments of the present invention to address the problems of prior art carrier particles per se, and in particular problems associated with liposome carriers, and to provide carrier particles exhibiting improved carrying properties for the payload biomolecule.

According to a first aspect of the present invention, there is provided a carrier particle arranged in use to encapsulate and carry a payload molecule to a target biological environment, the particle comprising an internal cavity in which a payload molecule is contained, the cavity being surrounded by a perm-selective hydrogel layer, wherein the payload molecule is capable of being active when the particle is at least adjacent the target biological environment.

The inventors have found that the carrier particle according to the first aspect of the invention is useful for targeting the payload molecule to a particular target biological environment, which may be a body fluid, or a specific tissue. Hence, the target biological environment may be a body fluid with circulation extended to substantially all of the body such as blood or lymph, or a body fluid with limited circulation, such as intraperitoneal or synovial fluids.

In a specific tissue, the target biological environment may be a cell or a group of cells. It is preferred that the target biological environment is outside a cell, i.e. in the extracellular matrix, or at the surface of the cell. Hence, by the term “at least adjacent the target biological environment”, it will be appreciated that the payload molecule is capable of being biochemically active when the particle is sufficiently close to the target biological environment, for example, the cell or group of cells, and preferably outside the cell or cells.

The group of cells may constitute a tissue or, for example, a cancerous body. The carrier particle may be used for performing biochemical conversions, or for the in vivo production of pharmacologically active components, or imaging agents. For example, the carrier particle may be used to locally perform chemical transformations of inert pro-drugs into corresponding active components, for example, in chemotherapeutics. In addition, the carrier particle may be used in the targeted delivery of an active compound towards tumoral masses.

Accordingly, the carrier particle comprises three primary components, namely: (i) the perm-selective hydrogel layer, which encapsulates (ii) the payload molecule, which is contained within the (iii) internal cavity. The perm-selective nature of the hydrogel layer enables the payload molecule to be active and functional when the carrier particle is at least adjacent the biological environment, i.e. the payload molecule is active when encapsulated by an intact hydrogel layer.

The hydrogel layer may comprise an organic and/or inorganic polymer. However, it is preferred that the hydrogel layer comprises substantially an organic polymer. Preferably, the hydrogel layer is substantially hydrophilic.

The hydrogel layer may comprise a plurality of polymer chains that are interconnected via cross-linkages. In the absence of cross-linkages, the polymer chains, which are preferably substantially hydrophilic, tend to diffuse in water and the carrier particle would be completely solubilised. Hence, the polymer chains may be connected through physical and/or covalent cross-links. However, in a preferred embodiment, the polymer chains are interconnected by covalent cross-links.

The presence of cross-links influences both the mechanical and permeability properties of the hydrogel layer. High cross-linking densities increase the layer's elastic modulus, and decrease the layer's permeability. In addition, the cross-links determine the formation of a network of polymer chains in the hydrogel layer, whose average mesh size can cause a Molecular Weight Cut-Off (MWCO) effect in the layer's permeability. By the term “average mesh size”, we mean the average size of pores in the polymer network hydrogel layer.

Molecules with a size larger than that of the average mesh size of the layer cannot diffuse through the layer, while permeation is possible for molecules with a smaller size. Hence, preferably, the particle's hydrogel layer is substantially permeable to molecules of small to moderate molecular size, but is substantially non-permeable to larger molecules. Advantageously, the carrier particle in accordance with the invention encapsulates and therefore protects the payload molecule from deleterious interactions or reactions, but allows favourable ones to occur.

By small to moderate molecular size, we mean molecules having a molecular size of less than 5 nm, more preferably, less than 3 nm, and most preferably, less than 2 nm. By large molecules, we mean molecules having a molecular size of greater than 5 nm, and preferably greater than 10 nm. The above measurements are given as the widest diameter of the molecule.

The effect of the hydrogel layer being substantially permeable to some molecules (small to medium size), but substantially impermeable to other molecules (large size) is referred to herein as size perm-selectivity. Hence, by the term “perm-selective”, we mean the hydrogel layer has a size-selective permeability. It is most preferred that the hydrogel layer is arranged to allow molecules smaller than 5 nm in diameter to pass therethrough, and to prevent or block molecules larger than 5 nm in diameter from passing therethrough.

Additionally, the hydrogel layer may be permeable to substrates with a certain chemical composition, or electrical charge, and not to others. For example, the hydrogel layer may present a net electrical charge that disfavours the approach and the permeation of molecules bearing a net electrical charge of same sign. For example, the net electrical charge of the hydrogel layer may be positive, which would repel molecules having a net positive charge. This effect is referred to herein as composition perm-selectivity. Hence, by the term “perm-selective”, we also mean the hydrogel layer has a composition-selective permeability.

It is especially preferred that the particle has both a size-selective permeability and composition selective permeability. With such dual permeability, the carrier particle is able to protect the encapsulated payload molecule and control the size, weight and chemical composition of molecules, which pass through the hydrogel layer, thereby affording controllable protection to the encapsulated payload molecule.

Preferably, the hydrogel layer comprises polymer chains resulting from a polymerisation reaction of one or more monomers, more preferably two or more monomers and most preferably two or more monomers one of them providing physical or chemical cross-links. Preferably, the hydrogel layer comprises polymer chains resulting from a living polymerisation mechanism reaction. The term living polymerisation refers to the absence or the negligible presence of termination reactions, an event that makes it possible to precisely control the molecular weight of polymers, and thus in this case the thickness of the hydrogel layer. Preferably, the monomers are water-soluble or water-dispersible monomers.

The perm-selectivity of the hydrogel layer may be controlled by carefully choosing suitable monomers for the polymerisation reaction. This is because the perm-selectivity of the layer is determined by the size of pores (i.e. the mesh size) extending therethrough, and because the pore sizes in the layer are determined a) by the molar fraction of the monomers that are capable to produce (physical or chemical) cross-links, b) by the number of cross-links that each of these monomers is able to produce, b) by the distances between the cross-links in the monomer structure. Hence, it is preferred that the polymers are cross-linked with a defined and controllable mesh-size in order to provide the desired size perm-selectivity, and which can be varied in composition in order to provide the desired composition perm-selectivity.

Suitable monomers that could be used to produce the hydrogel layer will be known to the skilled technician. Suitable monomers may provide a polymer structure as defined by Formula I below:

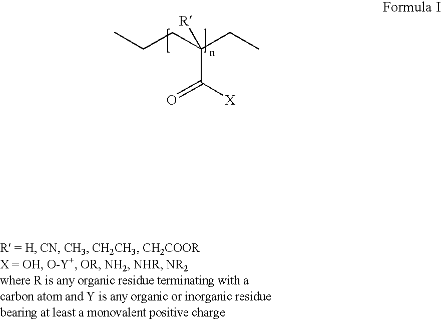

For example, suitable monomers may include acrylic, methacrylic or vinylic monomers. The polymer chains may be provided by polyacrylic, or polymethacrylic derivatives. The polymer chains may be provided by polyolefins (such as poly(vinyl pyrrolidone), poly(N-vinyl formamide), poly(vinyl alcohol), poly(N-vinyl imidazole), poly(2- or 4-vinyl pyridine) and the copolymers of all these units), polyoxazolines, polyethers, polysulfides, polysulfoxides, polysulfones, polyamines, polyammonium ions, and polyamides.

The length of the polymer chains relates to their degree of polymerisation, which determines the thickness of the hydrogel layer, which in turn influences the mechanical and transport properties of the layer. Thicker hydrogel layers have higher resistance to shear and require a longer time for a molecule or compound to permeate through them. Assuming an ideal coil conformation of the polymer chains and employing the Flory statistical theory, the thickness increases roughly linearly with the square root of the degree of polymerisation in the hydrogel layer.

In preferred embodiments, the polymer chains have degrees of polymerisation in the range 10²-10⁶ or more, which can correspond to thicknesses in the range of 10-500 nm, more preferably 50-300 nm and most preferably 100-200 nm. The degree of polymerisation is the number of monomer molecules making up the polymer chain. Such dimensions of the layer are preferred for providing protection to the active payload molecule in the internal cavity, but still allowing the perm-selective nature of the layer to be maintained. Preferably, the polymerisation reaction, which forms the hydrogel layer polymer, allows accurate control over the structural stability and perm-selective action of the layer. Hence, the carrier particle has a controllable hydrogel layer thickness.

This above-mentioned property may be obtained by applying a living polymerisation, such as Atom Transfer Radical Polymerisation (ATRP) onto a template molecule or matrix, which is described in more detail in respect of the second aspect of the invention (see below), and the Examples. It is preferred that the process takes place in a water environment, in order to avoid irreversible denaturation of the payload molecule.

For example, if the monomers used are 2-hydroxyethyl methacrylate (monofunctional monomer) and ethylene glycol dimethacrylate (difunctional monomer, therefore capable of forming chemical cross-links) in a 3:1 ratio, the polymer chains have cross-links each fourth monomeric unit (1 cross-link every 8 atoms) and the spacer between polymer chains is constituted by a chain of 6 atoms. This would therefore result in a hydrogel layer in which the pore sizes are approximately 0.5-0.7 nm, depending on the composition and ionic strength of the solvent, which is preferably a water mixture.

The repeating units of monomers in the polymer chains of the hydrogel layer may comprise at least one side chain, which may have a structural and/or functional role. However, it will be appreciated that not all the repeating units must present side chains.

For example, structural side chains may comprise groups with a well-defined hydrophilicity, which may contribute to the swelling degree of the hydrogel layer. Structural side chains may also provide a net charge to the hydrogel layer, which may favour absorption and permeation of oppositely charged chemical compounds, while disfavour absorption and permeation of chemical compounds bearing the same charge.

In preferred embodiments of the invention, structural side chains of the polymer may comprise sites for permanent cross-links based on the presence of covalent bonds or physical interactions. The density of both types of cross-links determines the mechanical and the perm-selective properties of the hydrogel layer. For example, a high cross-linking density determines a high elastic modulus of the hydrogel layer, i.e. a higher resistance to shear, compression and elongation stresses, and low permeability.

The polymer chains in the hydrogel layer may be substantially permanently and covalently cross-linked by spacer segments. The skilled technician will be aware of the chemical composition of the links between suitable spacer segments and polymer chains. For example, when acrylic or methacrylic polymer chains are used, ester or amide groups will be present at the connection between the spacer segments and the polymer chains.

The polymer chains in the hydrogel layer may be substantially permanently and physically cross-linked by spacer segments. The spacer segments can interact with two or more polymer chains and these interactions provide the cross-linking effect. For example, spacer segments bearing permanent electrostatic charges can provide permanent cross-links interacting with polymer chains bearing permanent opposite electrostatic charges. Spacer segments capable of forming multiple and asymmetric hydrogen bonding can provide permanent cross-links based on hydrogen bonding with complementary groups in the polymer chains.

The spacer segments may comprise an oligomeric or polymeric structure, such as a oligo- or poly(ether), (ester), (amide) or other structures that will be apparent to someone of skill in the art. However, spacer segments without oligomeric or polymeric structure may also be used.

The spacer segments may be linear, connecting at least two polymer chains, or branched, connecting at least three polymer chains. Examples of suitable spacer segments are illustrated in FIG. 19. Such spacer segments are listed by way of example only, and a number of variations (in the kind of polymer chain, in the functionality, size and chemical composition of the cross-linker) will be apparent to someone of skill in the art. At a comparable density of spacer segments, branched cross-links provide a higher cross-linking density and smaller mesh size and therefore higher modulus and lower permeability (or lower MWCO) of the hydrogel layer.

Comparing two spacer segments with the same degree of branching, a higher cross-linking density is provided by shorter segments, because of the shorter distance between polymer chains. The length of the segment increases with increasing degree of oligomerisation, increasing also the mesh size of the network.

The polymer chains in the hydrogel layer may be substantially permanently and physically cross-linked by direct interactions between structural side chains. For example, side chains bearing opposite permanent electrostatic charges may provide permanent cross-links based on electrostatic attractions. Side chains capable of forming multiple and complementary hydrogen bonding may provide permanent cross-links based on hydrogen bonding. Highly hydrophobic side chains may provide cross-links based on hydrophobic aggregation, and these cross-links are stable in the absence of solvents for the hydrophobic groups. The mesh size of the polymer network can therefore be modulated by varying the number of cross-linking side chains per polymer chain, the branching of these cross-linking units and their length.

Preferably, the average mesh size of the hydrogel layer is approximately 0.1 nm-50 nm, more preferably, approximately 0.5 nm-20 nm and most preferably, approximately 1 nm-10 nm. It is preferred that the average mesh size about 1 nm, 2 nm, 3 nm, or 4 nm. However, in a preferred embodiment, the mesh size is about 5 nm.

Functional side chains may present groups in the polymer of the hydrogel layer that are responsive to environmental conditions and that may be involved in reversible cross-linking. For example, these groups may vary their hydrophilicity and/or their association behaviour, and therefore the water content and/or mesh size of the hydrogel layer in response to variations of pH, ionic strength or composition, temperature or redox potential. Changes in the hydrogel layer water content and/or mesh size can cause swelling or shrinkage of the layer, and may influence its mechanical and transport properties. At a higher water concentration, the elastic modulus of the hydrogel layer decreases and the permeability increases. More particularly, by increasing the water content and/or mesh size, a molecule with a given molecular size can require a shorter time to travel through the layer. If the hydrogel layer cross-linking depends solely on the association behaviour of functional side chains, the modification of their hydrophilicity and/or association behaviour can solubilize the hydrogel layer, and thus all of the carrier particle.

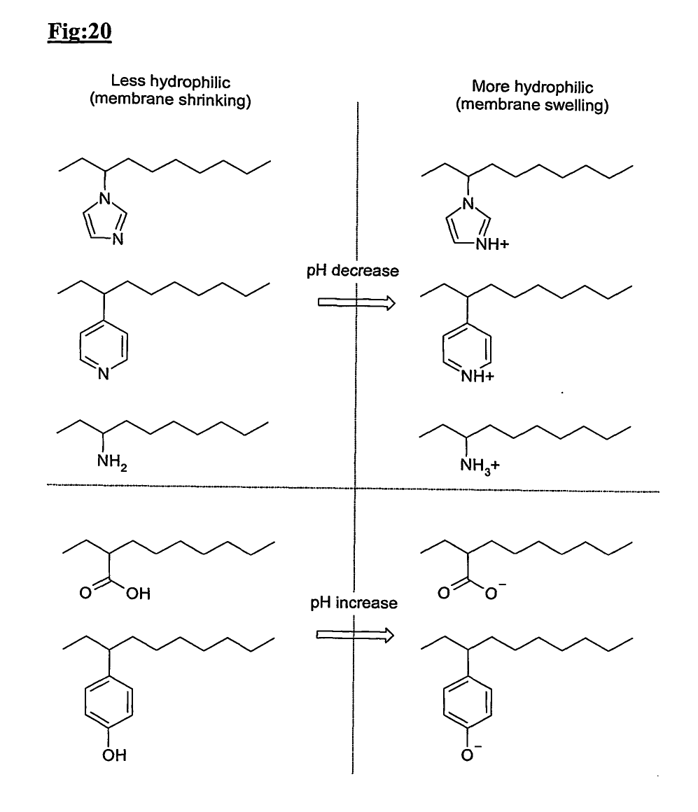

The polymer of the hydrogel layer, and more preferably its functional side chains, may comprise at least one group, which influences the layer's pH sensitivity. The pH sensitivity may be provided by groups that undergo an equilibrium of protonation/deprotonation, where charged species are more hydrophilic than neutral ones. A list of structures amenable to such equilibria is shown in FIG. 20. However, it will be appreciated that a variety of other groups may be used and will be known to those skilled in the art.

The polymer of the hydrogel layer, and more preferably its functional side chains, may also comprise at least one group, which influences the layer's sensitivity to ionic strength and composition. For example, sensitivity to ionic strength and composition may be provided by groups that undergo inter-chain association-dissociation phenomena (i.e. formation of reversible cross-links), in a fashion dependent on the number and on the nature of the surrounding ions. For example, multivalent ions, for example, group 2 alkali earth metal ions, such as calcium ions, may interact with more than one carboxylic group, with the ion occupying a ‘bridge’ position between two polymer chains. Increasing the multivalent ion concentration can promote interchain association, which causes a decrease in hydrophilicity and a shrinkage of the hydrogel layer. However, it will be appreciated that a variety of other groups may be used and will be known to those skilled in the art.

The polymer of the hydrogel layer, and more preferably its functional side chains, may also comprise at least one group, which influences the layer's sensitivity to temperature. For example, sensitivity to temperature may be provided by groups that undergo inter-chain temperature-dependent inter-chain association-dissociation phenomena (i.e. formation of reversible cross-links). For example, these side chains are composed by oligomers or polymers that present a Lower Critical Solubility Temperature in water, such as poly(ethylene glycol) homopolymers or block copolymers, poly(N-isopropyl acrylamide), poly(2-ethyl 2-oxazoline), poly(methyl vinyl ether), poly(N-vinyl caprolactam). However, it will be appreciated that this list provides only a number of examples, and it is not supposed to be exhaustive.

The polymer of the hydrogel layer, and more preferably its functional side chains, may also comprise at least one group, which influences the layer's sensitivity to redox potential. For example, sensitivity to redox potential may be provided by groups that undergo redox-dependent inter-chain association-dissociation phenomena (i.e. formation of reversible cross-links). For example, the hydrogel layer may be cross-linked partially or solely due to the presence of disulfides that bridge side chains in different polymer chains. The presence of, for example, reducing agents may convert them to thiols, or to asymmetric disulfides, determining a decrease of the cross-linking density and an increase of the hydrogel mesh size. If all hydrogel cross-links are based on disulfides, their cleavage may result in the solubilisation of the entire hydrogel, and thus of the carrier particle.

The polymer of the hydrogel layer may also comprise at least one group, which determines resistance to protein adsorption. Resistance to protein adsorption and absence of specific binding groups allow to confer to the carrier particle resistance to cell adhesion. For example, the hydrogel layer polymer may comprise at least one protein-repellant monomer, for example, a monomer containing at least one PEG chain. In the final hydrogel, PEG chains may be present as side chains or as spacer segments in the cross-links. Monomers containing tertiary amides, such as N,N-dimethyl acrylamide or N-vinyl pyrrolidone, or ether groups, such as 2-methoxyethyl or N-morpholin acrylate or methacrylate, may also be used. Monomers containing oligomeric or polymeric chains of the monomeric units mentioned above may also be used.

It will be appreciated that the polymer of the hydrogel layer, and more preferably, its functional side chains, may comprise any combination or all of the above-mentioned properties, i.e. pH sensitivity; ionic strength sensitivity; temperature sensitivity; redox potential sensitivity; and/or protein adsorption, depending on the final composition perm-selectivity of the carrier particle required, and this will be determined by the intended use of the carrier particle.

Preferably, the carrier particle in accordance with the invention is produced by the following steps:

(i) supporting or embedding the payload molecule in a matrix;

(ii) producing the polymeric hydrogel layer encapsulating the matrix; and

(iii) dissolving the matrix, to thereby produce the carrier particle.

Because the matrix is dissolved after the hydrogel layer has been produced around its surface, it is known as a sacrificial template. The size of the matrix determines the ultimate size of the internal cavity of the carrier particle.

When the monomer bearing the protein-resistant group(s) is polymerised with other monomers, it is possible to obtain hydrogel layers with preferential localization of the protein-resistant groups, for example, on the external surface where they can better express their function. The living polymerisation mechanism makes it possible to obtain ‘blocky’ structures that originate from an interface with the sacrificial template and terminate at the interface with the water solution. In a preferred embodiment, the ‘blocky’ polymer contains protein-resistant groups in its late block(s), which in the carrier particles are preferentially located close to the interface with the external water environment.

For example, on the sacrificial template, it is first polymerized 2-hydroxyethyl methacrylate (monofunctional monomer) alone or in mixture ethylene glycol dimethacrylate (bifunctional monomer and covalent cross-linker) in 10:1 molar ratio. Then, at complete conversion of this first monomer mixture, a second mixture composed of PEG2000 (poly(ethylene glycol) with MW=2000) monoacrylate alone or in mixture with PEG 570 diacrylate in 3:1 molar ratio, is added and polymerizes preferentially on the external surface of the hydrogel.

The polymer of the hydrogel layer may also comprise at least one group, which is adapted to present a biological recognition ligand for an active targeting or imaging action and/or a fluorescent label for detection. The fluorescent label may display an environmentally sensitive fluorescence, which is developed, increased, decreased or removed in response to an external stimulus, such as the presence of selected ions or substrates, or the REDOX potential of the environment.

The introduction of biological recognition ligands or fluorescent labels may be performed in two ways. A first method comprises the preparation of the hydrogel layer from one monomer bearing one or more reactive groups or from a mixture of monomers where at least one bears one or more reactive groups. The reactive groups are then successively used for the covalent binding to biological recognition ligands or fluorescent labels. A second method comprises the preparation of the hydrogel layer from one or more reactive groups or from a mixture of monomers where at least one bears one or more biological recognition ligands or fluorescent labels.

In the first method, the reactive groups may be present already in the chemical structure of the monomer. Therefore, the reactive groups must remain unaltered during polymerisation. Alternatively, the reactive groups may be generated on the polymer via deprotection or post-functionalization reactions. Typical preferred reactive groups may be carboxylic acids, amine-reactive esters (such as p-nitrophenyl, pentafluorphenyl, N-hydroxysuccinimidyl esters), primary and secondary amines, vicinal diols, thiols, thiol-reactive groups (such as 2-bromo esters, 2-iodo esters, maleimides, and acrylic, methacrylic or itaconic esters or amide). Typically, the amine- and thiolreactive groups may be generated on the polymer. These groups may then be reacted to form amides, esters, acetals and ketals and thioethers with a variety of chemical functionalities that are apparent to those skilled of the art.

When the monomer bearing the reacting group(s) is polymerized with other monomers, it may be possible to obtain hydrogel layers with preferential localization of the reactive groups, for example on the external surface, or on the internal surface. The living polymerization mechanism makes it possible to obtain ‘blocky’ structures that originate from the interface with the sacrificial template and terminate at that with the water solution. In a preferred embodiment, the ‘blocky’ polymer contains reactive groups in its late block(s), which in the carrier particle, are preferentially located close to the interface with the external water environment. This situation facilitates their functionalization with high molecular weight ligands of fluorophores, which are introduced in the external space and do not diffuse through the hydrogel to reach the reactive groups.

This situation is preferred for applications where the biologically recognition ligands and the fluorescent labels are supposed to interact with and display sensitivity to systems that cannot easily diffuse through the hydrogel layer. For example, the polymerization may be conducted on the sacrificial template by first employing a monomer mixture constituted by 2-hydroxyethyl methacrylate and ethylene glycol dimethacrylate in 10:1 molar ratio. When an 80% conversion of these monomers is reached, an amount of the reactive group(s)-containing monomer is added. This amount determines the functionality of the carrier particle and depends on the application and on the detection method of the labelling groups.

In a preferred embodiment, each carrier particle contains about 10²-10⁵ reactive groups and thus an equivalent amount of labels in its final state. For example, a 1% volume dispersion of carrier particles of 500 nm in diameter contains roughly 10¹⁴ carrier particles per liter. If each particle contains 10³ labelling groups, the final concentration of fluorescent labels is roughly 0.1 μM.

In another preferred embodiment, the ‘blocky’ polymer contains reactive groups in its first block(s), which in the carrier particles are preferentially located close to the interface with the external water environment. This situation facilitates their interactions with the encapsulated payload molecule or the products of its activity. For example, if the payload compound's activity produces labelled (e.g. fluorescent) molecules that diffuse through the hydrogel layer, a fraction of them can interact with the reactive groups and become covalently incorporated within the hydrogel. In the absence of reactive groups, the labelling diffuses out of the carrier particles and is lost. This method is useful for imaging the carrier particles in relation with their activity, because only carrier particles with active payload are labelled (e.g. are fluorescent).

The payload molecule, which is encapsulated within the carrier particle may be any molecule, which has suitable activity with or against the target biological environment, for example, the target cell type. For example, the payload molecule may have catalytic activity. It is preferred that the compound has bioactive properties, i.e. the compound has a biological effect on reaching the target environment while being retained in the carrier particle. Examples of the compounds bioactive properties are enzymatic reactions that hydrolyse protecting groups and transform prodrugs into active drugs.

It is preferred that the payload molecule remains in an active state while it is encapsulated within the carrier particle. Therefore, the molecule may be a relatively large molecule so that it is unable to permeate through the hydrogel layer. Hence, it will be appreciated that the size of the encapsulated molecule used is dependent on the structure, and composition, and mesh size, of the hydrogel layer so that it is retained therein. Preferably, the encapsulated molecule has a molecular size greater than 3 nm across, more preferably, greater than 5 nm, and even more preferably, greater than 7 nm across. In especially preferred embodiment, the payload molecule has a molecular size greater than 10 nm.

For example, the payload molecule may be a dye, electrochemical mediator, peptide, protein, antibody, or enzyme. The payload molecule may be derivatized with oligo- or polymeric species. The derivatization may be necessary if any of the previously mentioned examples does not reach the required size for avoiding permeation through the hydrogel layer.

Examples of suitable enzymes, which may be encapsulated in the carrier particle, include E. Coli cytosine deaminase, a bacterial (and hence immunogenic) enzyme that can catalyse the conversion of 5-fluorocytosine into the chemotherapeutic 5-fluorouracile; E. Coli nitroreductase, a bacterial (and hence immunogenic) enzyme that can catalyse the reduction of aziridin-containign prodrugs such as 5-(aziridin-1-yl)-2,4-dinitrobenzamide into cytotoxic compounds; and β-glucuronidase, a bacterial (and hence immunogenic) enzyme that can catalyse the conversion of doxorubicin glucuronide into the chemotherapeutic doxorubicin. Other possible enzymes are Herpes simplex virus thymidine kinase, and deoxycytidine kinase.

Hence, for example, the carrier particle may encapsulate a potentially immunogenic enzyme, such that the perm-selective membrane protects the enzyme from high molecular weight dangerous compounds, such as mediators of the immune system responses (immunoglobulins, complement) or proteolytic enzymes, but at the same time allows the permeation of substrate molecules in to the cavity of the particle, thereby allowing the enzyme to perform its biochemical reactions. In addition, the perm-selectivity of the membrane is such that products of enzyme catalysis may be able to permeate through the hydrogel layer and out of the particle. Hence, the encapsulated payload molecule can remain active for extended periods of time. In addition, by way of example only, the carrier particle may encapsulate an engineered protein, which is shielded from proteases outside the particle, but can signal, for example, by development of a fluorescence emission, the presence of a specific low molecular weight ligand, which can diffuse inside the particle.

The perm-selectivity characteristic of the hydrogel layer of the carrier particle in accordance with the present invention has a major advantage over existing carriers, such as liposome carriers or of polymeric hollow nanoparticles, which do not have a perm-selective layer. Accordingly, because the outer membrane of a liposome or of a polymeric hollow nanoparticle is not perm-selective, it is not possible for small to medium sized molecules (for example, substrate molecules) to permeate in to the centre of the liposome, and interact with the payload molecule, such as an enzyme. In addition, it is not possible for product molecules to permeate out through the membrane away from the payload molecule, e.g. enzyme. A liposome carrier encapsulates the enzyme, thereby protecting it from deleterious compounds present outside the carrier, such as proteases, as it moves towards a target environment. However, because the membrane of a liposome is not perm-selective, the encapsulated enzyme is maintained in an inactive state, until the liposome reaches its target environment. Upon reaching the target environment, the liposome is disrupted or bursts, thereby releasing the enzyme, which is thereby activated because substrate molecules are now able to come into contact with it.

Hence, it will be appreciated that liposomes carry their payload molecule in an inactive state, and are only useful therapeutically when they reach their target and are able to deliver their load following disruption. Once the liposome has burst releasing the payload, deleterious molecules such as proteases are able to react with it, thereby quickly deactivating the payload. Therefore, the payload is only active for a short period of time. In contrast, the carrier particles according to the present invention carry and deliver their payload in an active state due to the perm-selective nature of the hydrogel layer, thereby regulating the access of molecules to them on the basis of size and/or chemical composition. Furthermore, the active payload hydrogel layer protects the payload, thereby increasing the half-life of the active payload molecule, and therefore the length of it's activity.

Preferably, the internal cavity in which the payload molecule is contained is substantially aqueous, and preferably contains water. An aqueous cavity enables molecules (e.g. substrate and product compounds) to easily pass through the hydrogel layer (which is also substantially aqueous), in both directions to and from the payload molecule, so that the payload may be maintained in an active state.

Preferably, the carrier particle is small enough such that it may be suspended in fluids in a body, for example, the bloodstream. Therefore, it is preferred that the particle is sufficiently small such that it can pass along the vasculature system of an individual's body, i.e. veins, arteries, and/or capillaries.

Suitable sizes of the carrier particle are approximately 50 nm-1000 nm, more preferably 100-750 nm, and most preferably, 200-500 nm. Preferably, the carrier particle is substantially spherical in shape. Hence, the dimensions given above are the average diameter of the carrier particle. However, depending on environmental conditions an original carrier having spherical geometry may be modified to an elliptical or biconcave lenticular one. For example, an initial spherical geometry can become elliptical, due to the action of shear forces, or biconcave, due to higher external osmotic pressure.

Hence, the carrier particle is substantially spherical in shape, and consists of a hydrogel layer surrounding a hollow internal cavity. Therefore, due to its physical and structural composition, the carrier particle is also referred to herein as a hollow hydrogel nanosphere (HHN). A HHNs' hydrogel layer has a thickness controllable in the range 10-500 nm. Furthermore, it surrounds an internal cavity of a diameter in the range of 20-500 nm, which is filled with an aqueous solution. In addition, the HHN is constituted by a cross-linked, water-swollen polymer, referred to herein as cross-linked hydrogel layer. In a preferred embodiment, the hydrogel water content can be as high as 99%. The hydrogel is a mainly elastic and easily deformable material, whose response to high mechanical stresses can determine significant changes in the geometry of the HHN.

According to a second aspect of the invention, there is provided a method of producing a carrier particle in accordance with the first aspect, the method comprising the steps of:

(i) contacting a support matrix with a payload molecule;

(ii) producing a hydrogel layer encapsulating the matrix; and

(iii) dissolving the matrix, to thereby produce a carrier particle.

Preferably, the support matrix is a substantially spherical structure, which acts as a template or precursor for the successive phase. The size of the matrix determines the ultimate size of an internal cavity of the resultant carrier particle being produced. Preferably, the matrix has an average size in the range of about 20-500nm. Hence, the diameter of the cavity is approximately 20-500 nm.

Preferably, the payload molecule is substantially embedded within the matrix. The payload molecule may comprise a bioactive compound, for example, water-soluble peptides or proteins, enzymes, polysaccharides, antibodies, or synthetic polymeric materials.

The matrix may be used for providing morphology and size to the final carrier particle, and is then dissolved. Hence, it is preferred that the matrix is a sacrificial template as discussed above. The matrix may comprise an oxide, which may be inorganic oxide, and may be prepared through a sol-gel method based on the hydrolysis of organic precursors. Preferably, the matrix is prepared via a sol-gel method in reverse emulsion (water-in-oil emulsion). The sol-gel process is based on the conversion of the organic precursors in to silicic acid, and its subsequent condensation and aggregation to form a SiOₓOH₍₄₋₂ₓ₎ network, as illustrated in FIG. 13. This process may be completely performed in situ, with a rate that depends on the pH and the composition of the mixture.

For example, the organic precursors may comprise alkoxysilanes, and more preferably tetraalkoxysilanes, such as tetramethoxy-, tetraethoxy- or tetrapropoxysilane or tetrakis hydroxyalkoxysilanes, such as tetrakis (2-hydroxypropoxy)silane.

In other embodiments, the matrix may comprise an organic polymer that undergoes a sol-gel process in water solution. This sol-gel process may be activated by the presence of a gelling agent. Preferably, the sol-gel process is based on (poly)electrolyte complexation, which is harmless for biomolecules. For example, a solution of sodium alginate may be mixed with a calcium- or barium-containing solution to provide a calcium or barium alginate gel. The kinetics of this process may be adjusted by using complexing agents of the divalent ions. Other polymer-based gelling compositions will be apparent to someone of skill on the art.

A reverse emulsion (water-in-oil) may be generated by mixing a water phase, containing the payload compound in a hydrophobic fluid, preferably together with an appropriate emulsifier. This method is useful for producing silica templates with diameter of <100 nm (see Example 1). However, the hydrolysis of tetraalkoxysilane may be separately performed in a water solution with a controlled pH. This solution may then be buffered at a physiologically acceptable pH, added to the payload molecule, and dispersed in the reverse emulsion.

The water phase may comprise pure water, or a water-based buffer solution, or a mixture of water with a polar solvent (such as methanol, N-methyl pyrrolidone, N,N-dimethyl formamide) at a pH, ionic strength and composition that do not harm the activity of the active payload molecule.

The hydrophobic fluid may comprise an aliphatic compound, for example, an alkane or alkene. The hydrophobic fluid may comprise an alkane with a linear or branched chain, or a mixture of alkanes with linear or branched chains, or a mixture of such alkanes with alkyl-aromatic compounds. For example, the hydrophobic fluid may comprise hexane, or hexane and octane, or hexane and isooctane.

The emulsifier may comprise any amphiphilic substance or mixture of substances characterized by an appropriate hydrophilic lipophilic balance (HLB) value for the stabilization of the reverse emulsions constituted by the named hydrophobic fluid and the water phase. For example, the emulsifier(s) may comprise nonyl phenol ethoxylates, such as the compounds of the Berol series, or other oligoethoxylated non-ionic surfactants or sorbitan mono-, di- and tri-oleates or -stearates. Other non-ionic, anionic or cationic emulsifiers may also be used alone or in mixture, if required, provided they are capable of stabilizing the reverse emulsion, for example, AOT (2-ethyl-1-hexyl)sulfosuccinate, and its mixtures with non-ionic surfactants.

For the preparation of inorganic matrices, preferably alkoxysilanes are introduced in to the mixture, and are hydrolyzed by the water phase, producing silicic acid, and finally a network of Si—O—Si covalent bonds thereby embedding the payload molecule therein. This process transforms the original water-containing droplets into silica nanoparticles.

Alternatively, the alkoxysilanes may be first partially or completely hydrolyzed to silicic acid in a water environment, using an appropriate pH for obtaining a quick reaction, e.g. pH>8, or pH<6. The silicic acid may be aggregated under the influence of peptides, for example, high lysine-peptides, high arginine-peptides, such as SSKKSGSYSGSKGSKRRIL, or other derivatives of silaffin-1, or phosphoproteins such as some of the derivatives of silaffin-2.

For the preparation of organic matrices, preferably the polymer solution and the gelling agent may be mixed and immediately added of emulsifier and hydrophobic phase to generate a reverse emulsion, which hardens to generate gel nanoparticles. For example, a sodium alginate solution may be pre-mixed with a calcium or barium-containing solution and then emulsified. Alternatively, the polymer may be first emulsified and then added of a gelling agent solution. For example, 0.7 ml of a 1% wt. high M (high mannuronic acid) sodium alginate solution, 0.1 mM in a macromolecular payload (e.g. an enzyme), may be first dispersed in 40 ml hexane with the help of a mixture of emulsifiers (2 g of Berol 26 and 2 g of Berol 267) and then added of 0.3 ml of 0.1 M solution of calcium chloride in a citrate buffer.

Once the payload compound has been embedded in the matrix, the hydrogel layer may then be produced therearound. Preferably, a polymerization reaction between monomer subunits produces the hydrogel layer. Preferably, the hydrogel layer is formed substantially around an outer surface of the sacrificial template. It is preferred that Surface Atom Transfer Radical Polymerisation is used to form the hydrogel polymer layer around the surface of the matrix or sacrifical template produced by step 1.

After preparation of the matrix precursor, the surface thereof may be functionalised with groups capable of initiating polymerisation, preferably, living polymerisation. Suitable functional groups will be known to the skilled technician, for example, sterically hindered 2-halo ester and amides and other halides, preferably bromides, that can initiate Atom Transfer Radical Polymerization. In other embodiments, other groups may be used, such as thiols and amines, for the initiation of living polymerizations with ring opening mechanism. Such functional groups may be introduced via polyelectrolyte surface deposition or a silanisation reaction. In the polyelectrolyte surface deposition, the sacrificial template, when dispersed in water or in water mixtures at acceptable pH for the payload molecules (6-8), bears a net electrostatic charge, for example, a negative charge for pure silica or for calcium or barium alginate, a positive charge for amino-functionalized silica.

A polymeric or oligomeric species bearing an opposite net charge may be permanently adsorbed only due to electrostatic attraction and therefore provides a convenient method for surface functionalization. Preferably, the oligomeric or polymeric species is cationic with a net charge of at least 1 charged group each 5 repeating unit and a molecular weight not exceeding 20,000 g/mol. The charged polymer may bear functional groups that decorate the template after the surface adsorption takes place. In a preferred embodiment, the functional groups may be initiators for the Atom Transfer Radical Polymerization (ATRP) of monomers present in a medium surrounding the sacrificial template. This medium may be water or a water-containing solution.

Suitable monomers may be used for the polymerisation reaction, and preferably produce a polymer structure defined by Formula I above and is illustrated in FIG. 16. Examples include water-soluble or water-dispersable acrylic, methacrylic or vinylic monomers, which form a chemically and/or physically cross-linked hydrophilic polymer layer, i.e. the hydrogel layer, on the surface of the template. In a preferred reaction, multifunctional monomers are used, in order to obtain a chemically cross-linked hydrogel layer.

The monomer mixture may contain at least one protein-repellent monomer, for example, containing at least one poly(ethylene glycol) chain. The monomer mixture may also contain at least one monomer that features chemical groups with a specific activity, for example, displaying ligands required for biological recognition, or displaying groups for facilitating or hindering the diffusion of specific molecules in the hydrogel layer, i.e. composition perm-selectivity. The polymer chains may be permanently and physically cross-linked by spacer segments.

Following addition of functional groups to the surface of the matrix, the surface of the matrix is then treated with silanizing agents containing initiating groups for surface ATRP. Once exposed to a solution containing the monomers and catalyst, polymerisation proceeds from the surface and generates a surface film of hydrogel on the surface of the matrix.

According to a third aspect, there is provided a precursor for a carrier particle according to the first aspect, the precursor comprising a matrix containing a payload molecule, the surface of the matrix being encapsulated by a hydrogel layer.

The matrix may be dissolved to produce a carrier particle in accordance with the invention by suitable means, which will be appreciated by the skilled technician, and will be largely determined by the composition of the sacrificial template or support matrix. Hence, the precursor provides a preferred vehicle in which the payload molecule may be contained or embedded, for example, for transportation. Prior to use of the carrier particle, the matrix may be dissolved to ‘activate’ the particle.

Preferably, the matrix is dissolved by fluoride treatment for silica templates. More preferably, the fluoride-containing solution contains also ammonia or ammonium ions, which accelerate the process. Most preferably, the solution is buffered at a physiologically acceptable pH (5.5-8), in order to avoid an increase in pH during the dissolution of silica.

In other preferred embodiments, the matrix may be dissolved by complexing agents for alginate templates. For example, the nanoparticles may be dialyzed in an EDTA- or citrate-containing solution, which extracts the calcium ions and fluidifies the core. After calcium extraction, alginate macromolecules may remain in the water cavity, due to the molecular weight cut-off effect of the hydrogel layer. They may be used for adjusting the viscosity and the osmotic pressure of the water cavity, in order to provide additional stability to the HHNs.

The resulting hollow container features a water cavity containing the payload molecule. The polymeric hydrogel layer thereby forms a spherical membrane in which the payload molecule is encapsulated, and is perm-selective.

According to a fourth aspect of the present invention, there is provided a carrier particle arranged in use to encapsulate and carry a payload molecule to a target biological environment, the particle comprising an internal cavity in which a payload molecule is contained, the cavity being surrounded by a perm-selective hydrogel layer, wherein the payload molecule is capable of being active when the particle is at least adjacent the target biological environment, for use as a medicament.

According to a fifth aspect of the invention, there is provided use of a carrier particle arranged in use to encapsulate and carry a payload molecule to a target biological environment, the particle comprising an internal cavity in which a payload molecule is contained, the cavity being surrounded by a perm-selective hydrogel layer, wherein the payload molecule is capable of being active when the particle is at least adjacent the target biological environment, for the manufacture of a medicament for the treatment of diseases having a leaky or incompletely formed capillary vasculature.

According a sixth aspect of the invention, there is provided a method of treating an individual suffering from a disease having a leaky or incompletely formed capillary vasculature, the method comprising administering to an individual in need of such treatment a therapeutically effective amount of a carrier particle arranged in use to encapsulate and carry a payload molecule to a target biological environment, the particle comprising an internal cavity in which a payload molecule is contained, the cavity being surrounded by a perm-selective hydrogel layer, wherein the payload molecule is capable of being active when the particle is at least adjacent the target biological environment.

It is preferred that the payload molecule is capable of being active and is also protected when the particle is at least adjacent the target biological environment. The protection is conferred by the encapsulating hydrogel layer around the payload molecule.

It is most preferred that the carrier particle is particularly useful for treating conditions in which the individual to be treated has tissues with a leaky or incompletely formed capillary vasculature. Examples of pathological situations having a leaky or incompletely formed capillary vasculature include most solid tumors and wound healing, above all in the process of scar formation (angiogenesis sites: where new blood vessels and capillaries are quickly created). Hence, the carrier particle offers new biomedical applications for use in treating such conditions.

In one application, the carrier particles may be administered in the bloodstream, and accumulate selectively in angiogenesis sites, for example, wounds and tumours. This may be through the Enhanced Permeation and Retention effect, typical of colloidal materials.

In another application, the carrier particles may be administered percutaneously in regions of limited or negligible lymphatic uptake and remain there to express their activity. Alternatively, they can be injected percutaneously in regions of good lymphatic uptake and express their activity in the lymphatic system for a period of up to 2-3 weeks, before being released in the bloodstream.

The payload molecule may be imunogenic for the individual to be treated. Therefore, the payload molecule is destroyed by the individual's immune system when not encapsulated. Hence, this provides a positive selection for the targeting of the carrier particle, and hence, payload molecule. For example, the payload molecule may be a bacterial enzyme (if the individual being treated is non-bacterial, for example, mammalian such as a human), which enzyme converts a prodrug into a drug, which drug would kill a cancer cell. If the hydrogel layer is disrupted before the particle reaches the cancer cell, the bacterial enzyme would be destroyed by the immune system of the mammal.

In a preferred embodiment, the carrier particle comprises an average outer diameter of approximately 500 nm, and average internal diameter of approximately 100 nm. The internal surface the hydrogel layer substantially comprises poly(2-hydroxyethyl methacrylate) or poly(2-hydroxyethyl methacrylate-co-ethylene glycol dimethacrylate) in a 10:1 molar ratio. The outer surface the hydrogel layer substantially comprises poly(PEG2000 acrylate) or poly(PEG2000 acrylate-co-PEG 570 diacrylate) in a 3:1 molar ratio. This composition ensures good mechanical stability, appropriate mesh size (in the range of about 2-3 nm), and resistance to protein adsorption.

The carrier particle preferably encapsulates a volume of about 10⁶ nm³ occupied by a 0.1 mM solution of β-glucuronidase, thus encapsulating an average of roughly 10 enzyme molecules per nanoparticle. A 1% v/v dispersion of carrier particle provides a concentration of enzyme of about 1 nmol/l.

This dispersion may be injected intravenously in a healthy individual with no significant leaky vasculature and has a half life of 4-7 days or more in blood circulation, with increasing accumulation in organs such as lungs, liver, kidneys and spleen. The injection in individuals presenting solid tumoral masses, for example subcutaneously implanted colon cancer, lung cancer, colon cancer, results in the selective accumulation of the dispersion such that 20-50% of the dosage accumulates in the tumour after about 2 days. After the second day, daily intravenous injections (for periods of 5-20 days) of doxorubicin glucuronide provide the substrate for the enzyme action, which is mostly localized in the tumoral mass, due to the selective accumulation. In this way, a high concentration gradient of the enzyme product (the chemotherapic doxorubicin) is obtained, with a maximum concentration at the tumoral site.

It will be appreciated that the carrier particle according to the present invention may be used in a monotherapy (i.e. use of the carrier particle according to the invention alone to prevent and/or treat diseases having a leaky or incompletely formed capillary vasculature). Alternatively, the carrier particle according to the invention may be used as an adjunct, or in combination with known therapies.

Carrier particles according to the invention may be formulated in a composition. The composition may have a number of different forms depending, in particular, on the manner in which the composition is to be used. Thus, for example, the composition may be in the form of a capsule, liquid, ointment, cream, gel, hydrogel, aerosol, spray, micelle, transdermal patch, or any other suitable form that may be administered to a person or animal in a hydrated or moist form. It will be appreciated that the vehicle of the composition should be one which is well tolerated by the subject to whom it is given, and preferably enables delivery of the carrier particle to a target tissue.

Compositions comprising the carrier particle according to the invention may be used in a number of ways. For instance, systemic administration may be required in which case the carrier particle may be contained within a composition that may, for example, be ingested orally in the form of a capsule or liquid. Preferably, the composition may be administered by injection into the blood stream. Injections may be intravenous (bolus or infusion) or subcutaneous (bolus or infusion). The composition may also be administered by inhalation (e.g. intranasally).

The carrier particle may also be incorporated within a slow or delayed release device. Such devices may, for example, be inserted on or under the skin, and the composition may be released over weeks or even months. Such devices may be particularly advantageous when long term treatment with a carrier particle according to the invention is required and which would normally require frequent administration (e.g. at least daily injection).

It will be appreciated that the amount or number of carrier particles that is required is influenced by the required amount of payload molecule encapsulated therein, and its biological activity and bioavailability, which in turn depends on the mode of administration, the physicochemical properties of the carrier particle employed and whether the carrier particle is being used as a monotherapy or in a combined therapy. The frequency of administration will also be influenced by the above-mentioned factors and particularly the half-life of the encapsulated payload and carrier particle, within the subject being treated.

Optimal dosages to be administered may be determined by those skilled in the art, and will vary with the payload molecule and carrier particle in use, the strength of the preparation, the mode of administration, and the advancement of the disease condition. Additional factors depending on the particular subject being treated will result in a need to adjust dosages, including subject age, weight, gender, diet, and time of administration.

Known procedures, such as those conventionally employed by the pharmaceutical industry (e.g. in vivo experimentation, clinical trials, etc.), may be used to establish specific formulations of the carrier particle according to the invention and precise therapeutic regimes (such as daily doses of the carrier particle and the frequency of administration).

It will be appreciated that the dose of carrier particle is highly dependent on the specific payload molecule being carried, and the target cell, and the disease being treated. However, generally, a daily dose of between 0.01 μg/kg of body weight and 0.5 g/kg of body weight of the carrier particle according to the invention may be used for the prevention and/or treatment of a disease with a leaky or incompletely formed capillary vasculature, depending upon which specific carrier particle and payload molecule is used. More preferably, the daily dose is between 0.01 mg/kg of body weight and 200 mg/kg of body weight, and most preferably, between approximately 1 mg/kg and 100 mg/kg.

Daily doses may be given as a single administration (e.g. a single daily injection). Alternatively, the carrier particle used may require administration twice or more times during a day. As an example, the carrier particle according to the invention may be administered as two (or more depending upon the severity of the condition) daily doses of between 5 mg and 7000 mg (i.e. assuming a body weight of 70 kg). A patient receiving treatment may take a first dose upon waking and then a second dose in the evening (if on a two dose regime) or at 3 or 4 hourly intervals thereafter. Alternatively, a slow release device may be used to provide optimal doses to a patient without the need to administer repeated doses.

This invention provides a pharmaceutical composition comprising a therapeutically effective amount of a carrier particle according to the invention and optionally a pharmaceutically acceptable vehicle. In one embodiment, the amount of the carrier particle is an amount from about 0.01 mg to about 800 mg. In another embodiment, the amount of the carrier particle is an amount from about 0.01 mg to about 500 mg. In another embodiment, the amount of the carrier particle is an amount from about 0.01 mg to about 250 mg. In another embodiment, the amount of the carrier particle is an amount from about 0.1 mg to about 60 mg. In another embodiment, the amount of the carrier particle is an amount from about 0.1 mg to about 20 mg.

This invention provides a process for making a pharmaceutical composition comprising combining a therapeutically effective amount of a carrier particle according to the invention and a pharmaceutically acceptable vehicle. A “therapeutically effective amount” is any amount of a carrier particle according to the invention which, when administered to a subject provides prevention and/or treatment of a disease with leaky or incompletely formed capillary vasculature. However, it will be appreciated that the type and amount of payload molecule in the carrier particle will contribute to the therapeutic efficacy of the particle. A “subject” is a vertebrate, mammal, domestic animal or human being.

A “pharmaceutically acceptable vehicle” as referred to herein is any physiological vehicle known to those of ordinary skill in the art useful in formulating pharmaceutical compositions.

In a preferred embodiment, the pharmaceutical vehicle is a liquid and the pharmaceutical composition is in the form of a solution. In a further embodiment, the pharmaceutical vehicle is a gel and the composition is in the form of a cream or the like.

Liquid vehicles are used in preparing solutions, suspensions, emulsions, syrups, elixirs and pressurized compositions. The carrier particle can be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellent.

Liquid pharmaceutical compositions which are sterile solutions or suspensions can be utilized by for example, intramuscular, intrathecal, epidural, intraperitoneal, subcutaneous, and particularly, intravenous injection. The carrier particle may be prepared as a sterile solid composition that may be dissolved or suspended at the time of administration using sterile water, saline, or other appropriate sterile injectable medium. Vehicles are intended to include necessary and inert binders, suspending agents, lubricants, flavourants, sweeteners, preservatives, dyes, and coatings.

The carrier particle according to the invention can be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like.

The carrier particle according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:

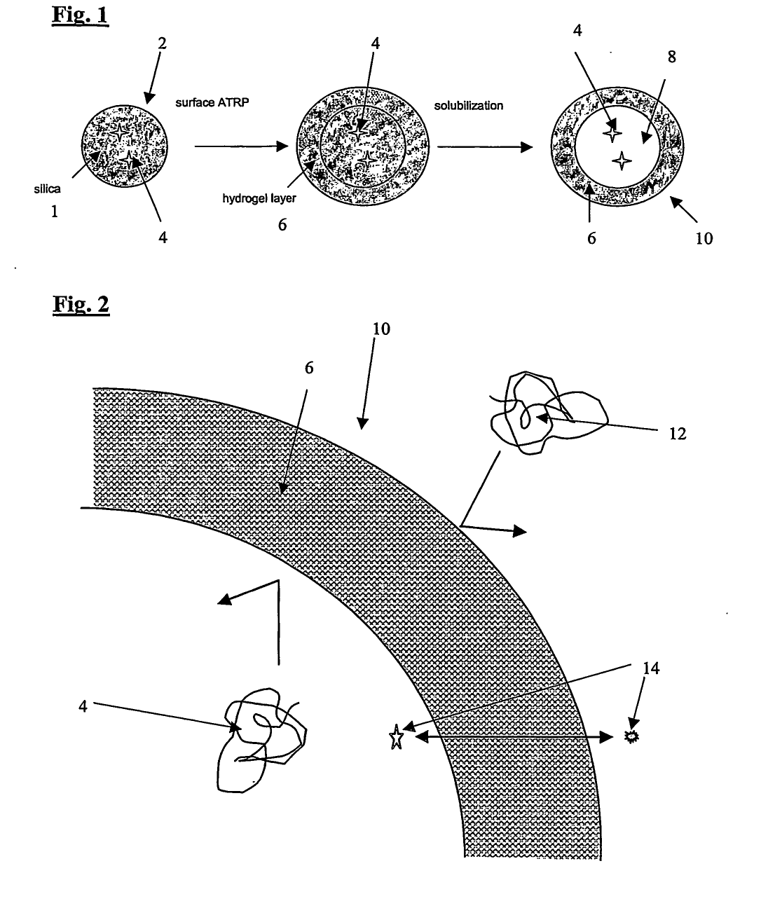

FIG. 1 is a schematic illustrating a sacrificial template prepared in the form of spherical construct eventually encapsulating an active material. Surface Atom Transfer Radical Polymerisation (ATRP) forms a polymer layer around the template. After solubilisation of the template, the active material is entrapped in the internal cavity because of difficult diffusion through the polymer layer;

FIG. 2 is a schematic illustrating an enzyme encapsulated in an embodiment of a carrier particle or hollow hydrogel nanosphere (HHN) according to the present invention;

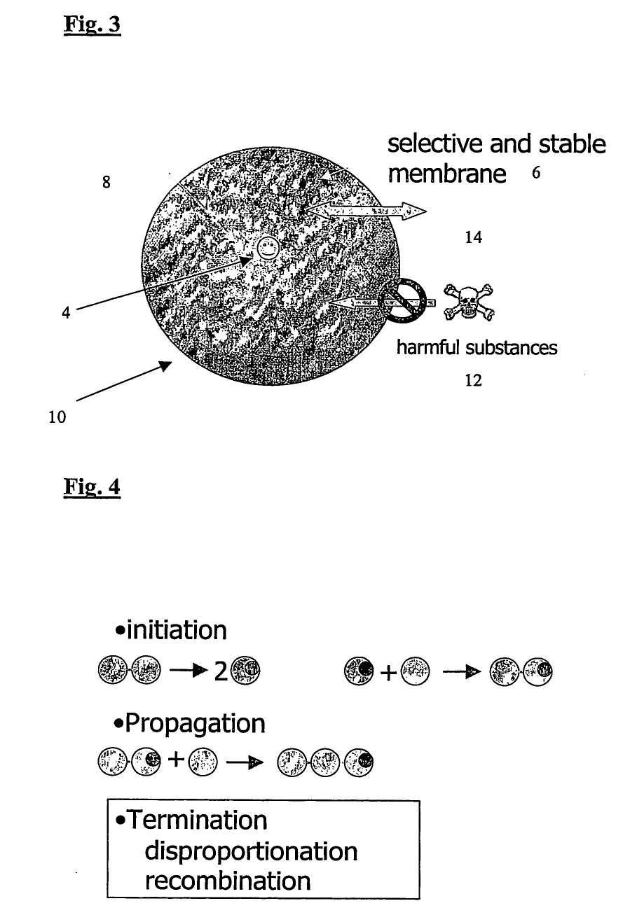

FIG. 3 is a schematic illustrating a further embodiment of the carrier particle containing an enzyme;

FIG. 4 is a schematic illustrating the mechanism of radical polymerisation;

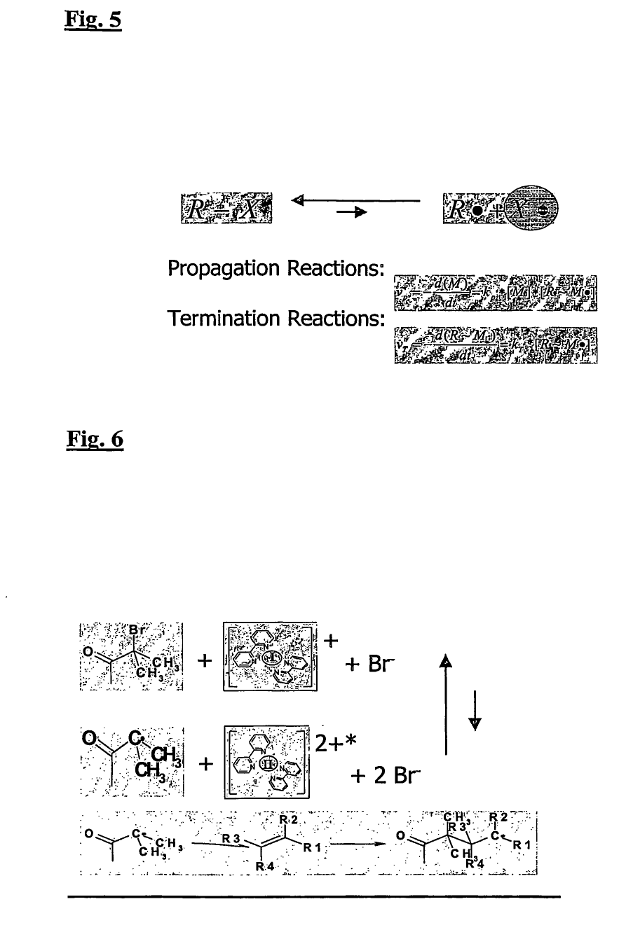

FIG. 5 is a schematic illustrating the mechanism of living controlled radical polymerisation;

FIG. 6 is a schematic illustrating Atom Transfer Radical Polymerisation (ATRP);

FIG. 7 is a schematic illustrating supported ATRP;

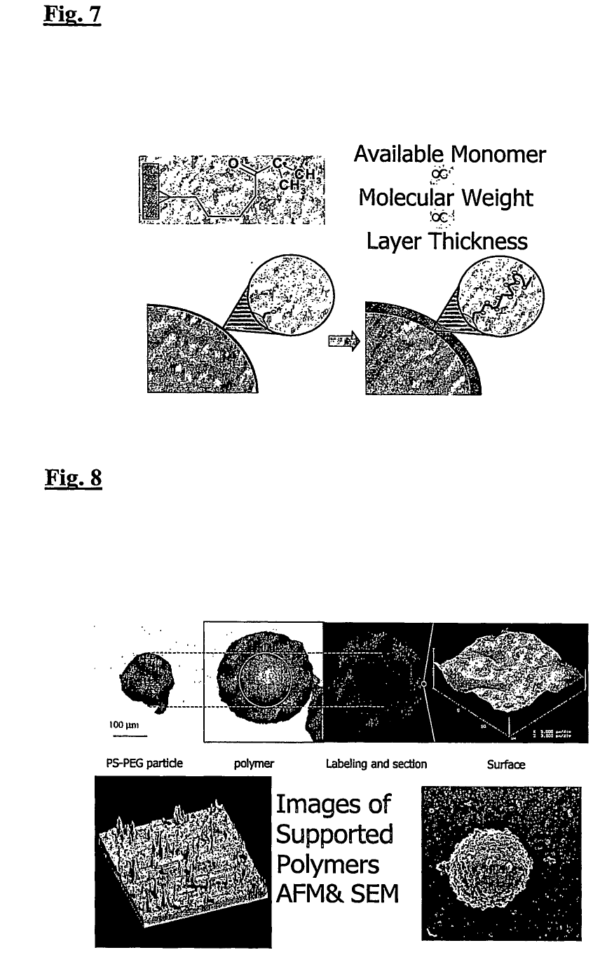

FIG. 8 is a schematic illustrating previous research;

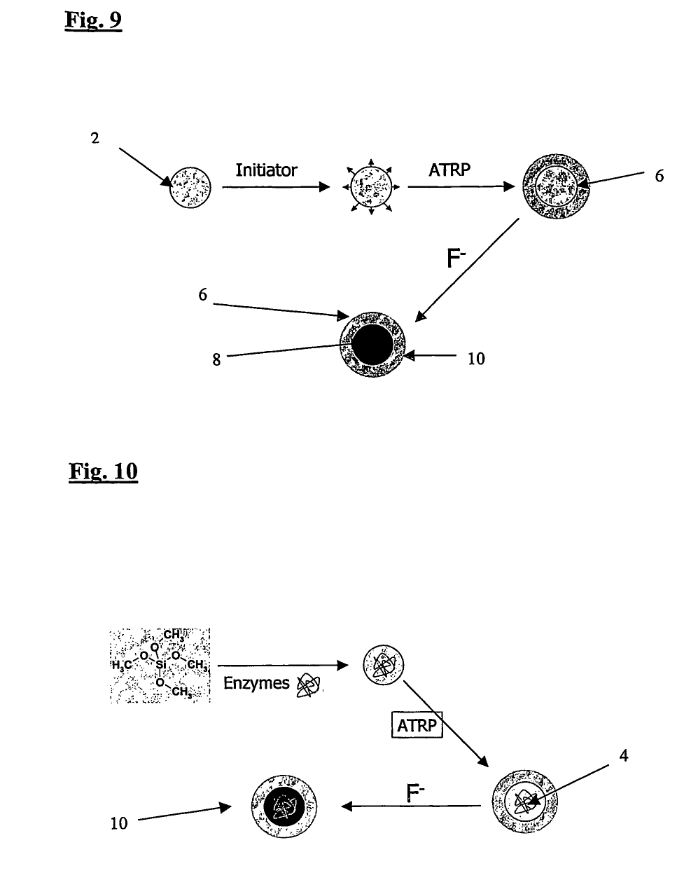

FIG. 9 is a schematic illustrating a further embodiment of a carrier particle or hollow nanosphere (HHN) in accordance with the invention;

FIG. 10 is a schematic illustrating encapsulated enzymes in a further embodiment of nanosphere in accordance with the invention;

FIG. 11 is a schematic view of a PEGylated liposome, which encapsulates a drug in its internal water cavity;

FIG. 12 is a schematic view of a further embodiment of a hollow hydrogel nanoparticle (HHN) that encapsulates an enzyme and regulates the access to it though the properties of the wall;

FIG. 13 is a schematic view of the generation of template nanoparticles in reverse emulsion;

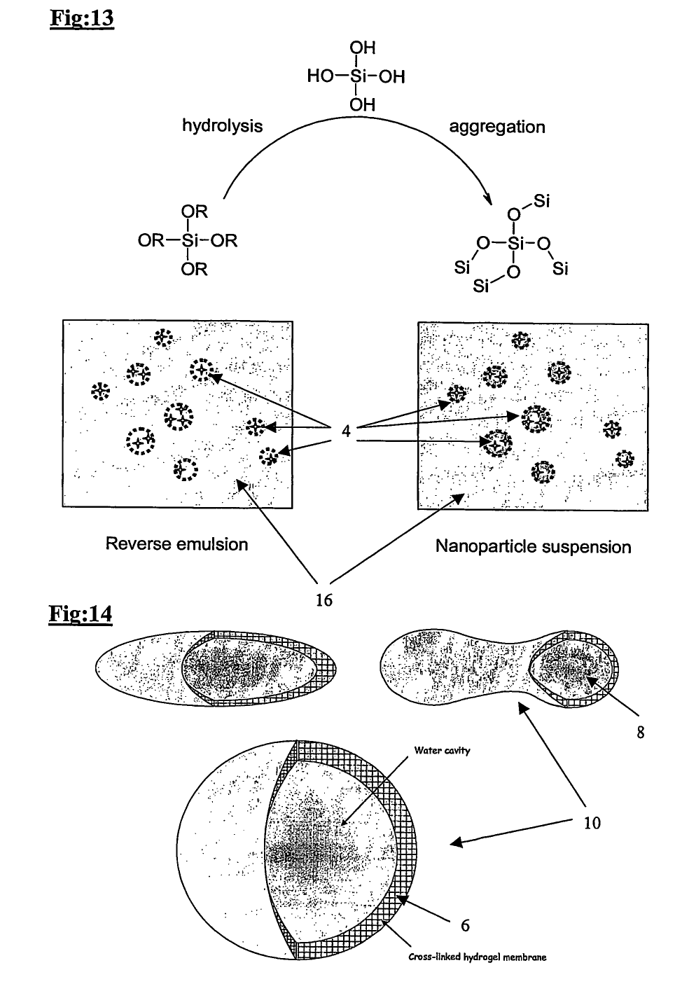

FIG. 14 is a schematic view of HHN structure and geometry;

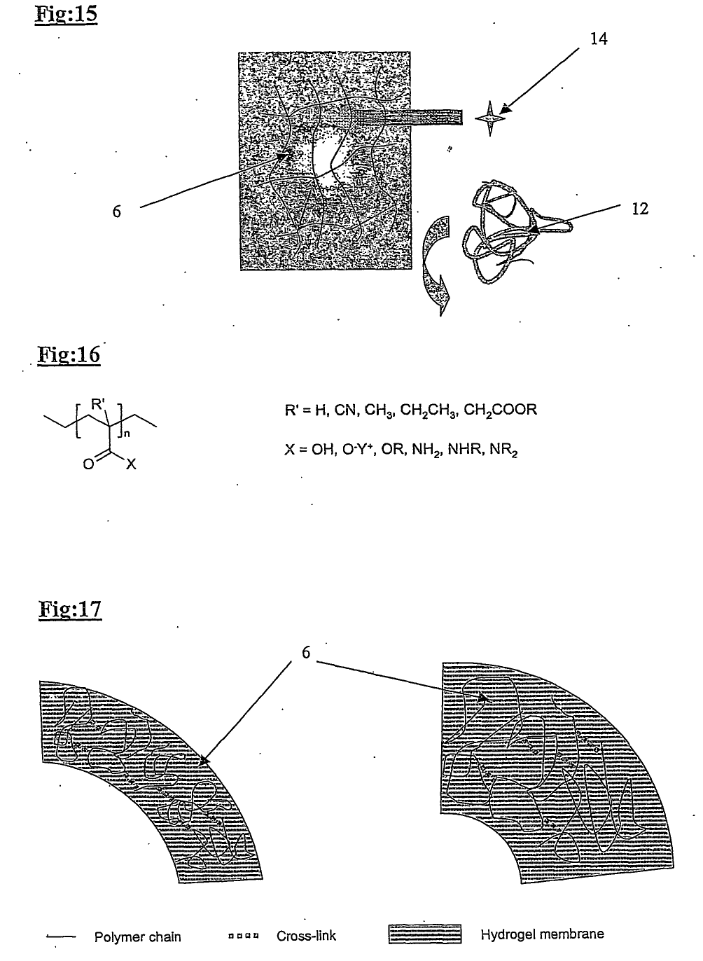

FIG. 15 is a schematic view showing the diffusion of molecules through a polymer network of an HHN;

FIG. 16 is a chemical structure of acrylic or methacrylic polymer chains for the HHN membrane composition.

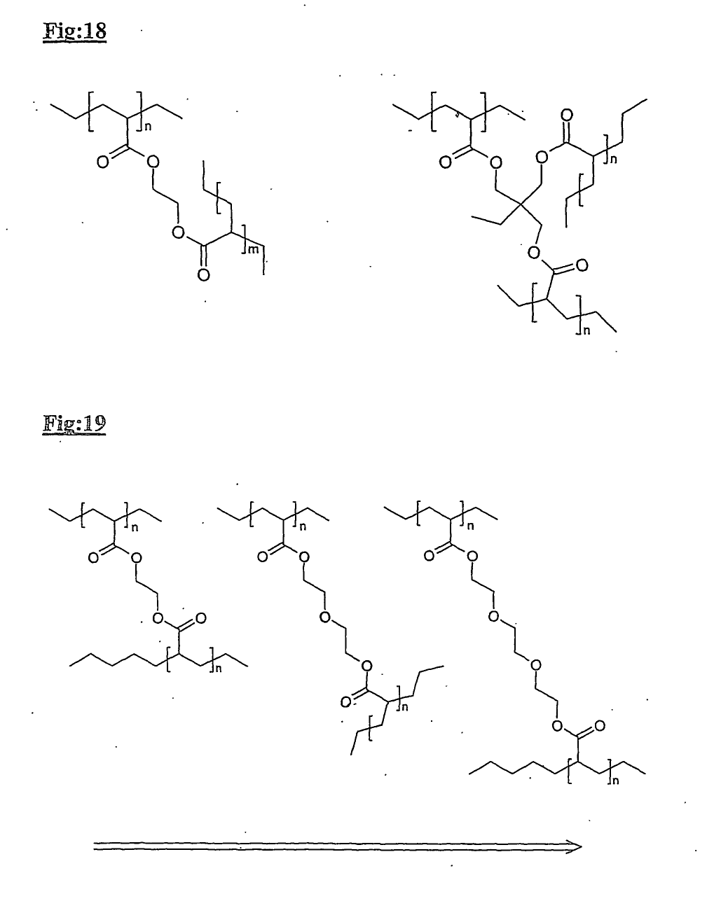

FIG. 17 is a schematic view of the length of the cross-linked polymer chains of the HHN membrane;

FIG. 18 are chemical structures of examples of bifunctional (left) and trifunctional (right) cross-linkers, bridging polyacrylate side chains;

FIG. 19 are chemical structures of examples of spacer segments derived from oligo(ethylene glycol) diacrylates with degree of oligomerization 1, 2 and 3;

FIG. 20 are chemical structures of examples of pH-sensitive groups that can be incorporated as side chains and of the effects arising from their protonation/deprotonation;

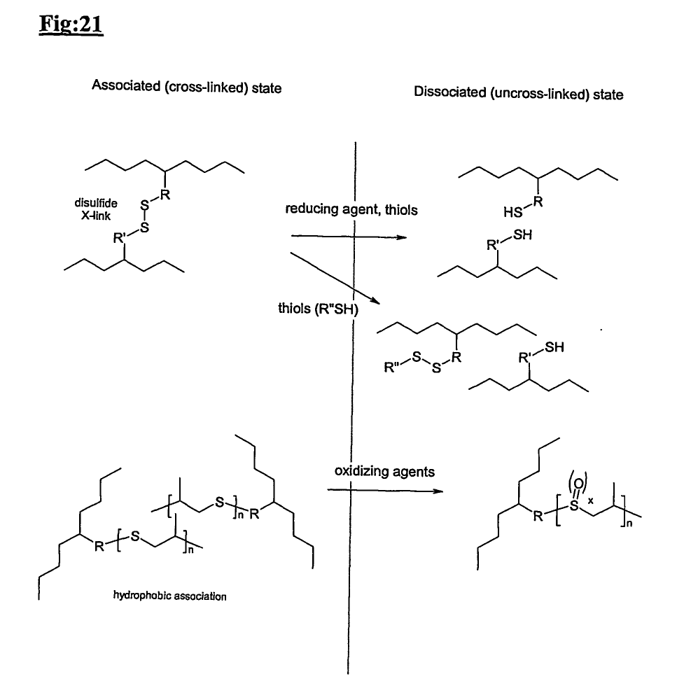

FIG. 21 are chemical structures of examples of REDOX potential sensitive groups that can be incorporated as groups;

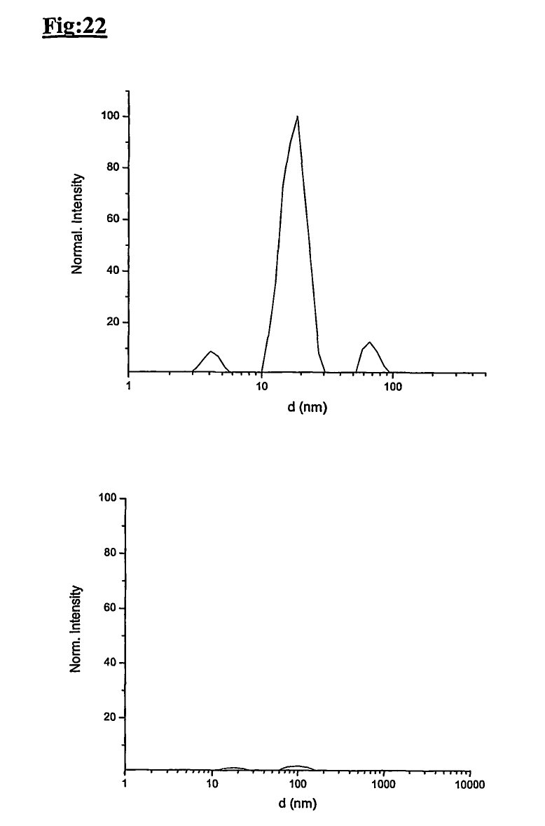

FIG. 22 are graphs showing on Left: unreacted Ludox nanoparticles. Right: Ludox nanoparticles at the same concentration after fluoride treatment.

Referring to FIG. 1, there is shown a schematic diagram illustrating the production of a carrier particle 10 in accordance with an embodiment of the present invention. Such carrier particles 10 are also known as hollow hydrogel nanoparticles or nanospheres (HHN) 10. Further details of the production of such HHNs 10 are illustrated in FIG. 9.

Specific examples for the manufacture of a carrier particle (HHN) 10 in accordance with the invention will now be described herein.

With reference to FIG. 1, the hollow hydrogel nanospheres 10 are prepared in three steps: (i) preparation of silica nanoparticles containing a payload molecule; (ii) use of these silica nanoparticles as a template for the production of a polymeric membrane therearound; and (iii) dissolution of the silica nanoparticles, to produce a resultant HHN 10. Each of these three steps will now be described in detail.

Step 1

In a first step, a spherical structure 2 is prepared, which acts as a template or precursor for the successive phase (step 2). This original spherical structure 2 has an average size in the range of about 20-500 nm, and is referred to as a sacrificial template 2. The sacrificial template 2 is used for providing morphology and size to a second structure, and is then dissolved.

Using established literature procedures (described hereinafter), silica nanoparticles 1 can be prepared in a wide range of sub-micron diameters, encapsulating biologically active substances 4. An example of a suitable biological substance is an enzyme, as shown in FIG. 1. These biological active compounds 4 retain their original activity, at least partially, in the final HHN 10.

In general, the process of step 1 or producing the template 2 is based on the hydrolysis and condensation of silicon alkoxides. The methodology employed for carrying out this process can determine the dimensions of the template 2, and, ultimately, that of the resultant HHN 10. The sacrificial template 2 is composed of an inorganic oxide and is prepared through a sol-gel method based on the hydrolysis of organic precursors. For example, in one embodiment, the organic precursors are tetraalkoxysilanes.

The sol-gel process is based on the conversion of the organic precursors in to silicic acid, and its subsequent condensation and aggregation to form a SiOₓOH₍₄₋₂ₓ₎ network, as illustrated in FIG. 13. This process can be completely performed in situ, with a rate that depends on the pH and the composition of the mixture. Alternatively, the tetraalkoxysilanes can be first partially or completely hydrolyzed to silicic acid in a water environment, using an appropriate pH for obtaining a quick reaction, e.g. pH>8 or <6.

The obtained solution is then introduced in to the environment for aggregation and preparation of the template 2. For example, silicic acid can be aggregated under the influence of appropriately designed peptides. Alternatively, the tetraalkoxysilanes may be first partially or completely hydrolyzed to silicic acid in a water environment, using an appropriate pH for obtaining a quick reaction, e.g. pH>8, or pH<6. The silicic acid may be aggregated under the influence of peptides, for example, high lysine-peptides, high arginine-peptides, such as SSKKSGSYSGSKGSKRRIL, or other derivatives of silaffin-1, or phosphoproteins such as some of the derivatives of silaffin-2. The methodology employed for carrying out this process can determine the dimensions of the template 2, and ultimately, the HHN 10.

In more detail, the sacrificial template 2 is prepared via a sol-gel method in reverse emulsion (water-in-oil emulsion), which is based on the in situ hydrolysis of tetraalkoxysilanes, as illustrated in detail in FIG. 13. A reverse emulsion (water-in-oil) is generated by mixing a water phase, containing the biologically active components 4 in a hydrophobic fluid 16, together with an appropriate emulsifier. This method is useful for producing silica templates with diameter of <100 nm (see Example 1). In another embodiment, the hydrolysis of tetraalkoxysilane is separately performed in a water solution with a controlled pH. This solution is then buffered at a physiologically acceptable pH, added to sensitive bioactive materials 4 and dispersed in the reverse emulsion.

The water phase is composed of pure water, or a water-base buffer solution, or a mixture of water with a very polar solvent (such as methanol, N-methyl pyrrolidone, N,N-dimethyl formamide) at a pH, ionic strength and composition that do not harm the activity of the active payload molecule component 4. The active payload molecule 4 includes, by way of example only, water-soluble peptides or proteins, enzymes, polysaccharides, antibodies, or synthetic polymeric materials. The hydrophobic fluid is an alkane with a linear or branched chain, or a mixture of alkanes with linear or branched chains, or a mixture of such alkanes with alkyl-aromatic compounds.

The emulsifier is any amphiphilic substance or mixture of substances characterized by an appropriate HLB value for the stabilization of the reverse emulsions constituted by the named hydrophobic fluid and the water phase. For example, the emulsifier(s) is a nonyl phenol ethoxylate, such as the compounds of the Berol series. Other non-ionic, anionic or cationic emulsifiers can also be used alone or in mixture, if required.

As shown in FIG. 13, a tetraalkoxysilane is then introduced in to the mixture, and is hydrolyzed by the water phase, producing silicic acid, and finally a network of Si—O—Si covalent bonds thereby embedding the payload molecule 4 therein. This process transforms the original water-containing droplets into silica nanoparticles.

Step 2

In a second step, Surface Atom Transfer Radical Polymerisation is used to form a hydrogel polymer layer 6 around the surface of the sacrifical template 2 produced by step 1, as shown in FIG. 1. After preparation of the template precursor 2, the surface of the sacrificial template 2 is first functionalised with groups capable of initiating living polymerization. Referring to FIG. 7, these functional groups are introduced via polyelectrolyte surface deposition or a silanisation reaction. The functional groups are initiators for the Atom Transfer Radical Polymerization (ATRP) of monomers present in the medium surrounding the sacrificial template 2. This medium is water or a water-containing solution.

The monomers that can be used for the ATRP are water-soluble or water-dispersable acrylic, methacrylic or vinylic monomers, which form a chemically cross-linked hydrophilic polymer layer 6, i.e. a hydrogel layer, on the surface of the template 2. The formula of preferred monomers used in ATRP is illustrated in FIG. 16. In a preferred reaction, multifunctional monomers are used, in order to obtain a chemically cross-linked hydrogel layer 6.

For example, in one embodiment, the polymer hydrogel layer can comprise groups, which influence pH sensitivity thereof. A list of suitable structures is shown in FIG. 20. In another embodiment, the polymer hydrogel layer may also comprise groups, which influence the layer's sensitivity to redox potential. For example, the hydrogel layer may be cross-linked partially or solely due to the presence of disulfides that bridge side chains in different polymer chains. The presence of, for example, reducing agents may convert them to thiols, or to asymmetric disulfides as illustrated in FIG. 21, determining a decrease of the cross-linking density and an increase of the hydrogel mesh size.

In another embodiment, the monomer mixture contains at least one protein-repellent monomer, for example, containing at least one poly(ethylene glycol) chain. In another embodiment, the monomer mixture can also contain at least one monomer that features chemical groups with a specific activity, for example, displaying ligands required for biological recognition, or displaying groups for facilitating or hindering the diffusion of specific molecules in the hydrogel layer 6, i.e. composition perm-selectivity. In another embodiment, the polymer chains are permanently and physically cross-linked by spacer segments. Comparing two spacer segments with the same degree of branching, a higher cross-linking density is provided by shorter segments, because of the shorter distance between polymer chains as illustrated in FIG. 19. The length of the segment increases with increasing degree of oligomerisation, increasing also the mesh size of the network.

In Atom Transfer Radical Polymerization, herein referred to as ATRP, the value of the polymer molecular weight is directly linked to the amount of available monomers, due to the suppression of termination reactions. ATRP has been previously applied to the coating of surfaces (Bontempo, D. et. al. Macromol. Rapid Commun. 2002, 23, 417-422, P. Von Natzmer et al. Chem. Commun. 2003, 1600-1601), where the film thickness can be tuned with the monomer quantity. This specific embodiment of ATRP, which proceeds from surface-linked groups on the template 2, and uses monomers and a catalyst in solution, is referred to herein as ‘surface ATRP’.

Following addition of functional groups to the surface of the template 2, the surface of the sacrificial precursor template 2 is then treated with silanizing agents containing initiating groups for surface ATRP. Once exposed to a solution containing the monomers and catalyst, polymerisation proceeds from the surface and generates a surface film of hydrogel 6.

Referring to FIGS. 4-7, there are shown details of the specific mechanisms of ATRP used in accordance with embodiments of the invention. FIG. 4 illustrates steps involved with radical polymerisation, i.e. an initiation step, followed by a propagation step, followed by a termination step. FIG. 5 illustrates steps involved with living controlled radical polymerisation. FIG. 6 illustrates steps involved with ATRP. FIG. 7 illustrates steps involved with supported ATRP. Surface ATRP allows a fine control over the thickness of the polymer layer 6.

The number and distance between polymerisable groups in the monomer structure will determine the mesh size of the final hydrogel layer 6 of the HHN 10, and therefore, its molecular weight cut-off, i.e. the size-dependent diffusion properties of objects in the hydrogel layer 6, i.e. size perm-selectivity. The length of the polymer chains is related to their degree of polymerization and determines the thickness of the hydrogel, which influences the mechanical and transport properties of the membrane. Thicker membranes have higher resistance to shear and require longer time for an object to permeate them. In a preferred embodiment, the polymer chains have degrees of polymerization in the range 10²-10⁶ or more, which can correspond to thicknesses in the range of 10-500 nm as illustrated in FIG. 17.

Hence, surface ATRP (a) can be initiated by inorganic or organic surfaces; (b) can proceed in a water environment; and (c) stops only when all the monomer is consumed (no parasite reactions). The thickness of the hydrogel layer 6 can be controlled by the polymerisation time, and amount of monomer added. The size of the object composed of sacrificial template 2 and hydrogel layer 6, i.e. a core-shell nanoparticle, is approximately, 50-1,000 nm.

Step 3

After the preparation of the hydrogel layer 6 by ATRP, the sacrificial template 2 is dissolved, for example with a fluoride treatment for silica templates, as illustrated in FIGS. 1, 9 and 10. The polymeric hydrogel membrane 6 thereby forms a spherical membrane 10 in which the bioactive payload compound 4 (enzyme) is encapsulated. FIG. 14 shows different embodiments of the HHN 10. An initial spherical geometry (bottom of FIG. 14) can become elliptical (top left), due to the action of shear forces, or biconcave (top right), due to higher external osmotic pressure.

The resulting hollow container 10 features a water cavity 8 containing the active payload molecule 4, which cannot diffuse out of the hydrogel layer 6 due to its large size, but can be reached by low molecular weight compounds 14 present in the surrounding water medium, and which can permeate the hydrogel layer 6. This is illustrated in detail in FIG. 15.

Active payload biomolecules 4 of a high molecular weight can be incorporated in the sacrificial template 2. If the polymer membrane 6 has an appropriate molecular weight cut-off, after the solubilisation of the template 2, then these components 4 are entrapped in the cavity 8, and retain their biological activity.

Referring to FIGS. 2 and 12, there is shown the structure of an HHN 10 in greater detail, in particular the permselective nature of the hydrogel polymer membrane 6. The HHNs 10 are novel because of the preparation method used, i.e. the preparation method applies surface ATRP for the synthesis of the spherical hydrogel membrane 6. The use of ATRP in the manufacture of the HHNs 10 allows precise control over thickness, functionality and transport properties of the membrane 6. Molecules with a size larger than that of the mesh size of the hydrogel layer cannot diffuse through the layer, while permeation is possible for molecules with a smaller size as illustrated in FIG. 15.

The polymer layer 6 is cross-linked and acts as a membrane with a molecular weight cut-off, ie. permselectivity. Hence, referring to FIG. 2, there is shown an enzyme 4 being encapsulated in the water cavity 8 of a hydrogel hollow sphere 10. The properties of the membrane 6 of the nanosphere 10 are engineered (high thickness and small mesh size) for allowing free diffusion or permeation of low Molecular Weight compounds 14, such as substrates and products of the enzymatic activity, but not of the enzyme 4 itself out of the carrier 10, or of potentially harmful high molecular weight compounds 12 (such as proteolytic enzymes or immune system mediators such as antibodies) into the cavity 8.

Referring to FIGS. 3 and 10, there is shown a methodology for encapsulating enzymes 4 in an HHN 10. Encapsulated enzymes 4 can preserve their activity as indicated by the permselectivity, but are protected against an external hostile environment. The enzymes 4 are used in body fluids, where the hollow structure 10 protects them from foreign body reactions and increases their half-life.

Application of Hollow Hydrogel Nanospheres

The resultant HHNs 10 may be used in the targeted delivery towards tumoral masses of functional and immuno-protected enzymes. They may also be used to locally convert inert prodrugs into chemotherapeutics. In a preferred application, the hydrogel hollow spheres 10 are administered in the blood and accumulate selectively in angiogenesis sites (wounds, tumours) through the Enhanced Permeation and Retention effect, typical of colloidal materials. The carriers 10 may feature a polymeric, hydrophilic spherical membrane 6 surrounding an internal water cavity, where the active principles 4 will be entrapped.

At the site of accumulation, they can perform chemical transformations of prodrugs into the corresponding active components. Referring to FIGS. 3 and 10, the hydrogel hollow spheres 10 contain enzymes 4 such as cytosine deaminase for the conversion of 5-fluorocytosine into the chemotherapeutic 5-fluorouracile.

Use of HHNs for in vivo Biochemical Conversions

The HHNs 10 are used to encapsulate compounds or mixtures of active compounds 4 capable of carrying out biochemical conversions. These compounds 4 can be natural or engineered enzymes or compounds that have enzyme-mimicking activity, or any compound that has a catalytic activity in physiological conditions. The active compounds 4 are encapsulated in the sacrificial template during its synthesis and remain entrapped in the water cavity of the HHNs after the solubilization of the template.

The mesh size of the hydrogel membrane 6 is engineered to provide a value that avoids diffusion of the active compound 4 in the outer environment, but allows that of the substrates of the compound or of possibly co-active compounds, such as co-enzymes.

For example, for an enzyme such as E. coli nitroreductase, with molecular mass reportedly of 28,000 g/mol, the average mesh size of the hydrogel membrane is set at an average value of 5 nm by the use of an appropriate mixture of comonomers in the preparation of the HHN hydrogel layer (for example, poly(ethylene glycol) methacrylate, MW=750 and ethylene glycol dimethacrylate). This mesh size value allows for the diffusion of substrates and products (aziridin-containing aromatic compounds, MW<1,000, size≦2 nm), but not of immunoglobulins (MW≧100,000, size≧10 nm).

The active compound-containing HHNs 10 can be used in vivo for providing a long-term activity to the encapsulated material. For example, HHNs 10 with a protein repellent and non-adhesive hydrogel membrane can be injected in a living animal directly in the blood stream or percutaneously for favouring their uptake in lymphatic capillaries. The mesh size of the hydrogel membrane 6 is engineered to provide a value that a) avoids diffusion of the active compound in the fluids, b) avoids the diffusion of substances that can potentially hinder the compound's activity (proteolytically active enzymes, antibodies) into the water cavity, c) allow permeation of the substrates of the compound or of co-active compounds.

Such systems can be used for the controlled production of drugs in body fluids. The use of appropriate enzymes that are not present in significant quantities in the targeted animal allows for performing conversions only in presence of the HHNs.

Use of HHNs for Bio-Imaging

HHNs 10 can be used for encapsulating bioactive materials, whose spectral properties change upon biochemical events. For example, HHNs can be used for encapsulating engineered Green Fluorescent Protein mutants, whose fluorescence change upon binding events. It is possible to detect the binding of engineered GFP mutants to low molecular weight ligands present in body fluids by detecting changes in GFP fluorescence. At the same time any influence from binding of high molecular weight protein or nucleic acid to GFP or any enzymatic degradation of GFP is avoided. In a preferred embodiment, mutant GFP-encapsulating HHNs are exposed to a sample of body fluids and the concentration of the target ligand is measured by measuring the changes in GFP fluorescence, for example its decrease due to quenching.

### EXAMPLES

Using the following detailed examples, the inventors show how:

a) it is possible to obtain sacrificial template nanoparticles with a controlled diameter (example 1);
b) this method can be extended to the preparation of template nanoparticles that contain sensitive biological material, i.e. the payload molecule (example 2);
c) the template nanoparticles can be solubilised in mild conditions (example 3);
d) it is possible to modify the surface of the template nanoparticles in a way that they are decorated with groups capable of initiating polymerisation (example 4);
e) the template nanoparticles modified on the surface with initiator groups can be decorated with polymer films (example 5.A. 1) via surface-initiated polymerization. The film thickness is controllable, due to the living character of the polymerization (example 5.A.2). The films can be composed of linear or cross-linked polymers (example 5.B). This method provides core-shell nanoparticles.
f) the cores of the core-shell nanoparticles can be solubilized, without damaging the shells and providing hollow nanospheres (example 6)
g) sensitive biological material (the payload molecule) entrapped in template nanoparticles (as in point b)) remains encapsulated in the core-shell structures and also in the hollow nanospheres (example 2)

### Example 1

### Preparation of Silica Nanoparticles

A One-Step Synthesis of Silica Nanoparticles with Controlled Diameter in Reverse Emulsion.

4 g of a mixture of anionic surfactants (2 g of Berol 26 and 2 g Berol 267) were dissolved in 40 ml n-hexane. The solution was added of 1.0 ml of a basic water solution (0.05M NH₃, previously prepared by diluting 0.1 g of NH₃ 35% in 40 ml pure water). The mixture was kept under stirring to form a stable and transparent microemulsion (slight blueish opacity). 0.6 ml of tetraethoxysilane (2.7 mmol; d=0.934 g/ml) were then added and left under stirring at 400 rpm for 72 hr. 60 ml of toluene were then added to the microemulsion, determining a flocculation and precipitation, which was completed in 15 min.

The precipitate was separated from the organic phase by decantation and resuspended in 40 ml of n-hexane. The white precipitate became transparent after 20 min and was then separated and washed again with 40 ml of n-hexane. The gel-like transparent precipitate was dispersed in 10 ml of 10 mM PBS solution at pH=7.4, providing a white emulsion (PBS composition: 0.2 g KCl, 0.26 g NaH₂PO₄*2H₂O, 2.86 g Na₂HPO₄*12H₂O, 8 g NaCl dissolved in 11 H₂O). The n-hexane still contained in the dispersion was evaporated at the rotary evaporator (pressure decreasing in steps from 400 to 20 mbar, ambient temperature). When the organic solvent was completely removed, the suspension turned transparent. The suspension was finally dialyzed in PBS (pH=7.2, dialysis membranes with MWCO 10 kDa) for 3 days to remove surfactants and possible traces of organic solvent.

After filtration through 1 μm filter, Dynamic Light Scattering analysis revealed two peaks, one composed by surfactant micelles (average diameter≈10 nm), which can be removed during dialysis, the other by nanoparticles (d≈50÷60 nm).

Other conditions for different diameters:

| | Result |
|---|---|
| Composition of the reverse emulsion | (using 0.6 ml tetraethoxysilane) |
| 2 g AOT (dioctyl sulfosuccinate) in | Agglomeration during the |
| 40 ml hexane, 0.5 ml aqueous phase | reaction |
| (0.05M NH₃) | |
| 2 g Berol 26 in 40 ml hexane, 0.5 ml | Nanoparticles ˜10 nm |
| aqueous phase (0.05M NH₃) | |
| 4 g Berol 26 in 40 ml hexane, 0.5 ml | Nanoparticles ˜10 nm |
| aqueous phase (0.05M NH₃) | |
| 2 g Berol 26 and 2 g Berol 267 in 40 ml | Nanoparticles ˜10 nm |
| hexane, 0.5 ml aqueous phase | |
| (0.05M NH₃) | |
| 2 g Berol 26 and 2 g Berol 267 in 40 ml | Nanoparticles 50-60 nm |
| hexane, 1 ml aqueous phase (0.05M | |
| NH₃) | |
| 2 g Berol 26 and 2 g Berol 267 in 40 ml | Instability of the reverse |
| hexane, 1.2 ml aqueous phase | emulsion |
| (0.05M NH₃) | |

Berol 26 and Berol 267 have identical hydrophobic part (nonylphenyl) and differently sized hydrophilic part, composed of 4 units of ethylene glycol for Berol 26 and 8 units for Berol 267 (HLB=8.9 for Berol 26, 12.3 for Berol 267). Increasing the quantity of Berol 267 one increases the average HLB and also the size of the water domains. Below a ratio 26/267<0.8, the emulsion is unstable.

B Two-Step Synthesis of Silica Nanoparticles with Controlled Diameter in Reverse Emulsion. Example of pH=7 Buffered Synthesis.

1 ml of tetraethoxysilane was added to 1 ml of a 1 mM HCl water solution and vigorously mixed. Complete solubilization of tetraethoxysilane was reached after 2 hr, which was taken as the minimum time for its complete hydrolysis. 1 ml of 10 mM phosphate buffer saline solution at pH=8 was added, raising the pH of the resulting solution to 7.0. 4 g of a mixture of anionic surfactants (2 g of Berol 26 and 2 g Berol 267) were dissolved in 40 ml n-hexane; 1.0 ml of the previously hydrolyzed and buffered tetraethoxysilane solution was added and the resulting microemulsion was treated as described in Example 1A.

### Example 2

### Solubilization of Silica Nanoparticles with Fluoride-Containing Solutions

A fluoride containing solution was prepared dissolving in 100 ml of H₂O 1 g of ammonium fluoride, 1 g of sodium fluoride, 1.5 g of glacial acetic acid and 2 g of ammonium acetate. The final solution is 270 mM in NH₄F, 240 mM in NaF, 250 mM in acetic acid and 260 mM in ammonium acetate, with a final pH of 5.7. 100 μl of commercially available Ludox silica solution (34% wt., Aldrich) were diluted in 10 ml of H₂O and the resulting solution was loaded into a dialysis bag with an appropriate Molecular Weight Cut-Off for the containment of nanoparticles but the free circulation of low molecular weight, soluble products (MWCO=15,000).

The dialysis bag was exposed to the 100 ml of fluoride solution for 18 hr. At the end of the process the pH was still 5.7. The dialysis bag was then removed and exposed to pure water for 6 hours for removing silicates and fluorides, exchanging each hour the surrounding solution with pure water. The content of the dialysis bag was then analyzed at the Dynamic Light Scattering and compared with an unreacted Ludox suspension at the same concentration. The two plots were normalized by dividing the peak intensities by the average scattered intensity (500 k cps for the unreacted sample, 13 k cps for the reacted one) and showed the almost complete dissolution of the nanoparticles, as illustrated in FIG. 22.

### Example 3

### Incorporation of Proteic Material in Silica Nanoparticles

The procedure used in Example 1B was modified to ensure the stability of the proteic material. 100 μl of a 4.1 mg/ml solution of Green Fluorescent Protein was diluted in 1.0 ml of 10 mM phosphate buffer saline solution at pH=8 and then added to the hydrolyzed tetraethoxysilane solution. The experiment was then carried out as described in Example 1B and the incorporation of the protein was proofed by measuring the fluorescence of nanoparticles dispersions after dialysis (MWCO=200,000).

### Example 4

### Functionalization of Silica Nanoparticles with Groups Capable of Initiating a Living Polymerization from the Nanoparticles Surface

A. Functionalization by Adsorption of a Cationic Polymer (pCAT) that Contains Groups Capable of Initiating Atom Transfer Radical Polymerization

A.1 Synthesis of the Polymer

Step 1—Synthesis of the Polymer Backbone

9 ml of 2-(dimethylamino)ethyl methacrylate (DMA) (53 mmol) and 2 ml of 2-hydroxyethyl methacrylate (HEMA) (13 mmol) were dissolved in 10 ml methanol and the solution was vigorously degassed with nitrogen for 45 min. 0.215 ml of ethyl 2-bromoisobutyrate (1.47 mmol) was added under nitrogen flux at a temperature of 20° C.; the addition of 0.46 g of 2,2′-bipyridyl (bpy, 2.94 mmol) and 0.22 g of Cu^{I}Br (1.47 mmol) started the polymerization reaction. After 15 minutes 40 ml of freshly degassed methanol were added, in order to avoid gelation in the reacting solution.

After 18 hr the solution was exposed to air (colour turns from dark brown to green), run through a silica chromatographic column to remove copper and finally evaporated. The solid was then dissolved in 25 ml of THF, filtered through a second silica column and precipitated in hexane. After a second precipitation, the polymer was dried under vacuum, to yield 8 g of dry white powder (yield 80%), composed by 35% HEMA (from NMR analysis).

¹H-NMR (CDCl₃): δ=4.0 (—COOCH₂CH₂—, both HEMA and DMA), 3.75 (—COOCH₂CH₂OH, only HEMA), 2.5 (—COOCH₂CH₂N(CH₃)₂, only DMA), 2.25 (—COOCH₂CH₂N(CH₃)₂, only DMA), 1.7-1.9 (—CH₂—C(CH₃)—, both HEMA and DMA), 0.7-1.0 (—CH₂—C(CH₃)—, both HEMA and DMA) ppm

$$GPC \text{:} \overline{{M}_{n}} = 3 \text{,} 200 ; \frac{\overline{{M}_{w}}}{\overline{{M}_{n}}} = 1.3$$

Step 2—Insertion of Initiator Groups onto the Polymer Backbone

4 g of the polymer synthesized in Step 1 (approx. 6.75 mmol —OH) were dissolved under nitrogen in 100 ml of THF. The solution was added of 2.5 g of 4-dimethylamino pyridine (20.25 mmol) and 6.6 ml of triethylamine (47.25 mmol). 4.2 ml of 2-bromo-isobutyryl bromide (33.75 mmol) were dissolved in 10 ml of THF and dropped under nitrogen into the solution keeping the temperature at 0° C. by the means of an ice bath. A white salt (triethylammonium bromide) instantaneously formed. At the end of the addition, the suspension was warmed up at room temperature and kept in this condition overnight.

The suspended salts were filtered away and the THF solution evaporated at the rotary evaporator. The oily material was dissolved in 50 ml dichloromethane, added with a 10 excess of triethylamine and washed with saturated NaCl water solution. The solution was then dried over sodium sulphate and then precipitated in hexane to yield 5 g of slightly yellow solid (approx. quantitative yield), with a quantitative degree of substitution of OH groups (from NMR analysis).

¹H-NMR (CDCl₃): δ=4.3 (—COOCH₂CH₂OOCC(CH₃)₂Br, reacted HEMA), 4.15 (—COOCH₂CH₂OOCC(CH₃)₂Br, reacted HEMA), 4.0 (—COOCH₂CH₂N═, DMA), 2.5 (—COOCH₂CH₂N(CH₃)₂, DMA), 2.25 (—COOCH₂CH₂N(CH₃)₂, only DMA), 1.9 (—COOCH₂CH₂OOCC(CH₃)₂Br, reacted HEMA), 1.7-1.9 (—CH₂—C(CH₃)—, both HEMA and DMA), 0.7-1.0 (—CH₂—C(CH₃)—, both HEMA and DMA) ppm

Step 3—Insertion of Cationic Groups onto the Polymer Backbone (Synthesis of pCAT)

300 mg of the polymer synthesized in Step 2 (approx. 0.97 mmol of tertiary amines) were dissolved in 10 ml of distilled water. When 1 ml of methyl iodide (162 mmol) were added under stirring the solution became a milky suspension (very low solubility of methyl iodide in water), whose opacity diminished with time. After 24 hours 100 ml of acetonitrile were added, causing precipitation of the polymer. The addition of acetronitrile facilitates the removal of water via azeotropic distillation at the rotary evaporator (the ratio is above the azeotropic point, which is reached at 16% in water). When about 50 ml of liquid were left, additional 50 ml of acetonitrile were added and the suspension was finally evaporated to dryness. 400 g of solid yellowish material were recovered.

¹H-NMR (D₂O): 4.2-4.5 (—COOCH₂CH₂—, both HEMA and DMA; —COOCH₂CH₂OOCC(CH₃)₂Br, HEMA), 3.7-3.9 (—COOCH₂CH₂N(CH₃)₃⁺, only DMA), 3.2-3.3 (—COOCH₂CH₂N(CH₃)₃⁺, only DMA), 1.7-1.9 (—COOCH₂CH₂OOCC(CH₃)₂Br, HEMA and —CH₂—C(CH₃)—, both HEMA and DMA), 0.8-1.1 (—CH₂—C(CH₃)—, both HEMA and DMA) ppm

A.2 Demonstration that the Polymer (pCAT) is Active for Initiating ATRP in Solution

20 mg of pCAT (0.0035 mmol of Br groups) were dissolved in 2.5 ml of water and degassed for 20 min by nitrogen bubbling. At the same time a solution of the ATRP catalyst was separately prepared by dissolving 21 mg of Cu(I)Br (0.15 mmol) and 46 mg bipyridyl (0.30 mmol) in 2.5 ml methanol and degassing the solution for 20 min. 1 ml of freshly distilled HEMA was added under nitrogen to the water solution. The two solutions were mixed under nitrogen atmosphere. Three samples, each of 0.5 ml, were taken from the polymerizing solution at different times (15 min, 30 min, 120 min) and added of 10 ml of acetonitrile. The solvent mixture was then removed at the rotary evaporator.

The solids were re-dissolved in 5 ml of methanol, and the resulting solutions were passed through silicagel columns in order to remove copper, before being again evaporated at the rotary evaporator. Finally, the solids were dissolved in 1 ml of ethanol and precipitated in 10 ml of hexane. After drying under vacuum, the quantity of recovered polymer was measured: the increasing quantity of polymer with time (39 mg, 54 mg and 140 mg, respectively) ensured the living character of the polymerization in the investigated time interval.

A.3 Adsorption of pCAT on the Surface of Silica Nanoparticles

In a general experiment, a suspension of nanoparticles was slowly added to a solution containing a large excess of pCAT. The optimal concentration conditions must be found to avoid nanoparticles aggregation, agglomeration and possibly precipitation. We found that a pCAT concentration of at least 5 mg/ml provides good coating.

| | Nanoparticle | | |
|---|---|---|---|
| | concentration | pCAT | Nanoparticle final |
| Nanoparticles | (vol. %) | concentration | diameter |
| Ludox (˜20 nm) | 0.2 | 10 mg/ml | 70 nm |
| >> | >> | 5 mg/ml | 70 nm |
| >> | 0.3 | 2.5 mg/ml | >450 nm |
| | | | (agglomeration) |
| >> | 0.35 | 1.2 mg/ml | = (precipitation) |
| Custom-made | 2.5 | 5 mg/ml | 80 nm |
| nanoparticles | | | |
| (50-60 nm, see | | | |
| Example 1) | | | |
| >> | >> | 10 mg/ml | 80 nm |

### Example 5

### Polymerization from pCAT Adsorbed on Silica Nanoparticles

A Polymerization of a Monofunctional Monomer

A.1 Proof of Principle of Polymerizability

0.5 ml of a water dispersion of pCAT-coated nanoparticles, with average diameter=80 nm (60 nm before coating) at a concentration of 5% vol. and displaying a bromide group concentration of 3.4*10⁻³ mmol, was diluted with water to a final volume of 2.5 ml and degassed for 20 min by bubbling nitrogen. 21 mg of Cu(I)Br (0.15 mmol) and 46 mg of dipyridyl (2×0.15 mmol) were introduced in a vial under nitrogen and dissolved into 2.5 ml of previously degassed methanol. The nanoparticles water solution was then introduced under nitrogen atmosphere in the vial. 2 ml of the water/methanol solution were transferred to a second vial and added of 25 mg of pure 2-hydroxyethyl methacrylate (HEMA). After 3 hr of polymerization the sample was analyzed at the Dynamic Light Scattering and showed an increase in average diameter from 80 to 400 nm.

A.2 Proof that the Thickness of the Polymer Layer can be Controlled with Polymerization Time

3 ml of a water/methanol solution containing nanoparticles and inititators, prepared as described in Example 5.A1, were transferred to a vial and added of 0.5 ml (very big excess) of pure HEMA. The sample was analyzed at the Dynamic Light Scattering as a function of time, providing an increase in average diameter from 80 nm (t=0) to 170 nm (15 min), 200 nm (30 min). Flocculation of the sample (the nanoparticles are so big to coalesce) was recorded at t=2 hr.

B Polymerization of Monomer Mixtures Containing Multifunctional Monomers and Preparation of a Cross-Linked Hydrogel Layer

0.5 ml of a water dispersion of pCAT-coated nanoparticles, with Ø=80 nm (60 nm before coating) at a concentration of 5% vol. and displaying a bromide group concentration of 3.4*10⁻³ mmol, was diluted with water to a final volume of 2.5 ml, added of 0.2 g of poly(ethylene glycol) dimethacrylate (PEGDMA, Molecular Weight=870) and degassed for 20 min by bubbling nitrogen. 21 mg of Cu(I)Br (0.15 mmol) and 46 mg of dipyridyl (2×0.15 mmol) were introduced in a vial under nitrogen and dissolved into 2.5 ml of previously degassed methanol. 0.2 ml of HEMA was finally added.

The nanoparticles water solution was then introduced under nitrogen atmosphere in the vial. After 15 min of polymerization the sample was analyzed at the Dynamic Light Scattering and showed an increase in average diameter from 80 to 160 nm.

### Example 6

### Preparation of Hollow Nanospheres by Solubilization of the Sacrificial Template

Two samples of 2 ml each containing the polymerization solutions of the examples 5.A2 (30 min) and 5B were introduced in dialysis bags and exposed for 18 hr to 100 ml of fluoride-containing solution, following the same method of silica dissolution described in Example 2. The dialysis bags were then removed and exposed to pure water for 4 hr for removing silicates and fluorides, exchanging each hour the surrounding solution with pure water.

The core-shell silica-hydrogel nanoparticles originally displayed diameters of 200 and 160 nm, respectively for the polymerization of pure HEMA (Example 5.A2) and of HEMA/PEGDMA (Example 5.B). After fluoride treatment and dialysis purification, Dynamic Light Scattering analysis provided diameters of 450 and 300 nm, respectively, indicating the permanence of colloidal structures after the solubilization of the silica cores.

### SUMMARY

In summary, the invention relates to permselective carrier particles, otherwise known as hollow hydrogel nanospheres (HHNs). These hollow structures are produced by essentially three key steps: (a) preparation of silica nanoparticles in which a payload biomolecule is embedded; (b) use of these as a template for the production of the polymeric membrane by ATRP; (c) dissolution of the silica nanoparticles.

Hence, ATRP is used to provide a system for the encapsulation of sensitive and bioactive ppayload compounds, which are structured in (A) an internal water compartment 8 containing compound(s) 4 of interest; and (B) a hydrogel membrane 6 that isolates the water compartment 8 from the external environment in a size permselective way, and also a composition permselective way. The invention is based on the understanding of the techniques used for the production and characterisation of these systems such as analytical techniques such as fluorimetry (used to study labelled compounds), dynamic light scattering (for the characterization of the size of colloidal aggregates) and rheology (for the study of the agglomeration of colloidal suspensions).

Two main methods have been devised for the production of silica nanoparticles, both starting from organic precursors of silicon dioxide, and the tetraalkoxy silanes. The inventors have studied the influence of preparation variables such as the characteristics of reverse emulsions (type and concentration of the emulsifier, water-to-oil ratio) and the pre- or post-hydrolysis of silica precursors on the dimensions and stability of the nanoparticles as discussed in the examples.

Regarding surface-initiated polymerisation, the Atom Transfer Radical Polymerization was found to be a suitable mechanism for growing polymer chains from a surface in water environment. In contrast to most radical polymerisation techniques, ATRP shows negligible transfer of the polymerisation to solution and a living character. In this way, the thickness of the polymer layers 6 is directly dependent on the amount of monomers used, and very high values can be easily obtained.

The inventors studied the surface functionalisation of silica nanoparticles and the application of ATRP for the decoration of the nanoparticles 10 with hydrophilic polymer layers 6. Several monomers were applied, with the aim to study the dependence of the membrane transport properties and stability on its chemical composition. The work focused onto the dissolution step, optimizing the conditions for the use of fluoride ions in the solubilization of the silica cores; particular care has been devoted to the establishment of a method that avoids pH drifts during the reaction and maintains a physiologically acceptable acidity.

Payload molecules 4, e.g. enzymes were encapsulated in the silica nanoparticles 2 during the alkoxysilane condensation process. Their stability and activity in the free form were studied and compared to that after the solubilisation of the silica core 2. Advantages of the hollow hydrogel nanoparticles 10 reside in the permselectivity of the hydrogel layer 6. The polymerization of multifunctional hydrosoluble monomers in surface ATRP produces a hydrogel layer 6 with controlled thickness and mesh size. These two quantities determine the diffusion properties of molecules through the layer 6. Hence, the carrier molecules 10 in accordance with the invention have utility in the delivery of bioactive compounds to target sites in the body, for example, for use in cancer therapy.

Advantageously, the carrier particle 10 encapsulates and protects the payload molecule 4 from dangerous interactions, but allows for favourable ones to occur. The carrier particle 10 may be used to encapsulate potentially immunogenic enzymes, protecting them from an immune system reaction, but at the same time allowing them to perform biochemical transformations. The carrier particle 10 may be used to encapsulate engineered proteins, which are shielded by proteases but can signal (for example by development of a GFP).

The carriers' hydrogel layer 6 is permeable to substrates of small to moderate molecular size (ligands), but not to large molecules (proteases, antibodies), ie. size perm-selectivity. Alternatively, hydrogel layer is permeable to substrates with a certain chemical composition or electrical charge and not to others, i.e. composition perm-selectivity.

Recent journal literature reports methods for the preparation of hollow colloidal structures with a wall (or membrane) in principle amenable or modifiable to gather the above mentioned properties. Most commonly, these structures can be provided by methodologies that use a solid template as a core, which is solubilized at the end of the process, i.e. the sacrificial template. However, other hollow structures (liposomes) can also be used as precursors, or the wall can be produced by the means of interfacial using emulsions as templates.

In many methodologies, the water cavity is produced from a template that initially does not contain water. On the contrary, for ensuring the encapsulation of functional active components in embodiments of the present invention, the initial stage active components (the payload molecule 4) are encapsulated in the water-rich template.

Among the structures presented in literature, those obtained via polyelectrolyte coacervation or polymerization of conductive polymers on a sacrificial template, via other hollow templates, or via hydrogen bonding, provide very limited wall thicknesses (a few nm). On the contrary, for a good control over mechanical stability and perm-selective action, the HHN 10 in accordance with the first aspect of the invention, has higher and controllable wall thickness.

This last property has been obtained by applying a living polymerization, such as Atom Transfer Radical Polymerization (ATRP) onto a template. This methodology has normally been applied to prepare hydrophobic walls. On the contrary, in accordance with the first aspect of the invention, ATRP is used to provide hydrophilic walls, in order to favour the permeation of water-soluble molecules. Additionally, the ATRP process to takes place in a water environment, in order to avoid irreversible denaturation of the active components.

Furthermore, the hydrophilic walls are cross-linked with a defined and controllable mesh-size in order to provide size perm-selectivity, that can be varied in composition, in order to provide composition perm-selectivity.

Furthermore, the composition of the hydrophilic wall 6 reduces protein adsorption and/or cell adhesion, in order to reduce clearance rate and prolong circulation times in body fluids. The composition of the hydrophilic wall 6 reduces protein adsorption and simultaneously presents biological recognition ligands, in order to provide active targeting possibilities.

1-37. (canceled)

38. A carrier nanoparticle arranged in use to encapsulate and carry a payload molecule to a target biological environment, the nanoparticle comprising an internal cavity in which a payload molecule is contained, the cavity being surrounded by a substantially hydrophilic hydrogel layer, which comprises polymer chains resulting from a living polymerisation reaction such that the hydrogel layer has size-selective permeability, composition-selective permeability, or both, and wherein the payload molecule is capable of being active when the nanoparticle is at least adjacent the target biological environment.

39. A carrier nanoparticle according to claim 38, wherein the carrier nanoparticle is approximately 50 nm-750 nm in diameter.

40. A carrier nanoparticle according to claim 38, wherein the target biological environment is a body fluid with circulation extending to substantially all of the body such as blood or lymph, or a body fluid with limited circulation, such as intraperitoneal or synovial fluids, or a cell or a group of cells.

41. A carrier nanoparticle according to claim 38, wherein the hydrogel comprises a polymer structure as defined by Formula I below: 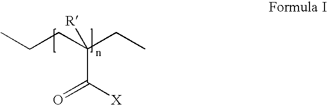
wherein R′=H, CN, CH₃, CH₂CH₃, or CH₂COOR,
X=OH, O⁻Y⁺, OR, NH₂, NHR, or NR₂,
R is any organic residue terminating with a carbon atom, and
Y is any organic or inorganic residue bearing at least a monovalent positive charge.

42. A carrier nanoparticle according to claim 38, wherein hydrogel layer has a thickness in the range of 10 nm-500 nm.

43. A carrier nanoparticle according to claim 38, wherein the polymer chains in the hydrogel layer are substantially permanently and physically cross-linked by spacer segments.

44. A carrier nanoparticle according to claim 43, wherein the spacer segments comprise an oligomeric or polymeric structure, such as a oligo- or poly(ether), (ester), or (amide).

45. A carrier nanoparticle according to claim 43, wherein the spacer segments are linear, connecting at least two polymer chains, or branched, connecting at least three polymer chains.

46. A carrier nanoparticle according to claim 38, wherein the average mesh size of the hydrogel layer is approximately 0.1 nm-5 nm.

47. A carrier nanoparticle according to claim 38, wherein the payload molecule is a dye, electrochemical mediator, peptide, protein, antibody, or enzyme.

48. A carrier nanoparticle according to claim 38, wherein the internal cavity in which the payload molecule is contained is substantially aqueous.

49. A carrier nanoparticle according to claim 38, wherein the carrier nanoparticle is approximately 100 nm-500 nm in diameter.

50. A method of producing a carrier nanoparticle according to claim 38, the method comprising the steps of:
(i) contacting a support matrix with a payload molecule;
(ii) producing a hydrogel layer encapsulating the matrix; and
(iii) dissolving the matrix, to thereby produce a carrier nanoparticle.

51. A method according to claim 50, wherein the payload molecule is substantially embedded within the matrix.

52. A method according to claim 50, wherein the hydrogel layer is produced by surface Atom Transfer Radical Polymerisation.

53. A method according to claim 50, wherein the support matrix is a silica sacrificial template.

54. A method according to claim 53, wherein the silica sacrificial template is dissolved by fluoride treatment.

55. A method according to claim 54, wherein the fluoride treatment comprises using a fluoride-containing solution, which contains ammonia or ammonium ions.

56. A carrier nanoparticle arranged in use to encapsulate and carry a payload molecule to a target biological environment, the nanoparticle comprising an internal cavity in which a payload molecule is contained, the cavity being surrounded by a substantially hydrophilic hydrogel layer, which comprises polymer chains resulting from a living polymerisation reaction such that the hydrogel layer has size-selective permeability, composition-selective permeability, or both, and wherein the payload molecule is capable of being active when the nanoparticle is at least adjacent the target biological environment, for use as a medicament.

57. Use of a carrier nanoparticle arranged in use to encapsulate and carry a payload molecule to a target biological environment, the nanoparticle comprising an internal cavity in which a payload molecule is contained, the cavity being surrounded by a substantially hydrophilic hydrogel layer, which comprises polymer chains resulting from a living polymerisation reaction such that the hydrogel layer has size-selective permeability, composition-selective permeability, or both, and wherein the payload molecule is capable of being active when the nanoparticle is at least adjacent the target biological environment, for the manufacture of a medicament for the treatment of diseases having a leaky or incompletely formed capillary vasculature.

58. A method of treating an individual suffering from a disease having a leaky or incompletely formed capillary vasculature, the method comprising administering to an individual in need of such treatment a therapeutically effective amount of a carrier nanoparticle arranged in use to encapsulate and carry a payload molecule to a target biological environment, the nanoparticle comprising an internal cavity in which a payload molecule is contained, the cavity being surrounded by a substantially hydrophilic hydrogel layer, which comprises polymer chains resulting from a living polymerisation reaction such that the hydrogel layer has size-selective permeability, composition-selective permeability, or both, and wherein the payload molecule is capable of being active when the nanoparticle is at least adjacent the target biological environment.

59. A method of treatment according to claim 58, wherein diseases having a leaky or incompletely formed capillary vasculature include tumors, and vasculature associated with wound healing.
